SEQUENCE LISTING (39 sequences)

SEQ ID NO: 1 - protein, 320 aa, Homo sapiens
  MISC_FEATURE: Human VSTM4 with predicted signal sequence
  MISC_FEATURE (1)..(23): Predicted signal sequence
MRLLALAAAALLARAPAPEVCAALNVTVSPGPVVDYLEGENATLLCHVSQKRRKDSLLAVRWFFAHSFDSQEALMVKMTKLRVVQYYGNFSRSAKRRRLRLLEEQRGALYRLSVLTLQPSDQGHYVCRVQEISRHRNKWTAWSNGSSATEMRVISLKASEESSFEKTKETWAFFEDLYVYAVLVCCVGILSILLFMLVIVWQSVFNKRKSRVRHYLVKCPQNSSGETVTSVTSLAPLQPKKGKRQKEKPDIPPAVPAKAPIAPTFHKPKLLKPQRKVTLPKIAEENLTYAELELIKPHRAAKGAPTSTVYAQILFEENKL

SEQ ID NO: 2 - protein, 180 aa, Artificial Sequence
  Human VSTM4 ECD (AA1-AA180) with predicted signal sequence
  MISC_FEATURE (1)..(23): Predicted signal sequence
MRLLALAAAALLARAPAPEVCAALNVTVSPGPVVDYLEGENATLLCHVSQKRRKDSLLAVRWFFAHSFDSQEALMVKMTKLRVVQYYGNFSRSAKRRRLRLLEEQRGALYRLSVLTLQPSDQGHYVCRVQEISRHRNKWTAWSNGSSATEMRVISLKASEESSFEKTKETWAFFEDLYVY

SEQ ID NO: 3 - protein, 157 aa, Artificial Sequence
  Human VSTM4 ECD (AA24-AA180) without predicted signal sequence
LNVTVSPGPVVDYLEGENATLLCHVSQKRRKDSLLAVRWFFAHSFDSQEALMVKMTKLRVVQYYGNFSRSAKRRRLRLLEEQRGALYRLSVLTLQPSDQGHYVCRVQEISRHRNKWTAWSNGSSATEMRVISLKASEESSFEKTKETWAFFEDLYVY

SEQ ID NO: 4 - protein, 132 aa, Artificial Sequence
  Human VSTM4 Ig-like domain (AA24-AA155)
LNVTVSPGPVVDYLEGENATLLCHVSQKRRKDSLLAVRWFFAHSFDSQEALMVKMTKLRVVQYYGNFSRSAKRRRLRLLEEQRGALYRLSVLTLQPSDQGHYVCRVQEISRHRNKWTAWSNGSSATEMRVIS

SEQ ID NO: 5 - protein, 107 aa, Artificial Sequence
  Human VSTM4 IgV domain (AA24-AA130)
LNVTVSPGPVVDYLEGENATLLCHVSQKRRKDSLLAVRWFFAHSFDSQEALMVKMTKLRVVQYYGNFSRSAKRRRLRLLEEQRGALYRLSVLTLQPSDQGHYVCRVQ

SEQ ID NO: 6 - protein, 319 aa, Mus musculus
  MISC_FEATURE: Mouse VSTM4 with predicted signal sequence
  MISC_FEATURE (1)..(23): Predicted signal sequence
MRLRLLALAAAVLLGPAPEVCGALNVTVSPGPVVDYLEGENATLLCHVSQKRRKDSLLAVRWFFAPDGSQEALMVKMTKLRIIQYYGNFSRTANQQRLRLLEERRGVLYRLSVLTLRPTDQGQYVCKVQEISKHRNKWTAWSNGSSATEMRVISLKAGEDSSFEKKKVTWAFFEDLYVYAVLVCCVGILSVLLFTLVIAWQSVFHKRKSRVRHYLVKCPQNSSGETVTSVTSLAPLQPQKGKRQKKKVDVPPAVPAKAPIATTFHKPKLLKPQRKVALPKITEENLTYAELELIKPHRAAKGVPTSTVYAQILFEENQL

SEQ ID NO: 7 - protein, 179 aa, Artificial Sequence
  Mouse VSTM4 ECD (AA1-AA179) with predicted signal sequence
  MISC_FEATURE (1)..(23): Predicted signal sequence
MRLRLLALAAAVLLGPAPEVCGALNVTVSPGPVVDYLEGENATLLCHVSQKRRKDSLLAVRWFFAPDGSQEALMVKMTKLRIIQYYGNFSRTANQQRLRLLEERRGVLYRLSVLTLRPTDQGQYVCKVQEISKHRNKWTAWSNGSSATEMRVISLKAGEDSSFEKKKVTWAFFEDLYVY

SEQ ID NO: 8 - protein, 156 aa, Artificial Sequence
  Mouse VSTM4 ECD (AA24-AA179) without predicted signal sequence
LNVTVSPGPVVDYLEGENATLLCHVSQKRRKDSLLAVRWFFAPDGSQEALMVKMTKLRIIQYYGNFSRTANQQRLRLLEERRGVLYRLSVLTLRPTDQGQYVCKVQEISKHRNKWTAWSNGSSATEMRVISLKAGEDSSFEKKKVTWAFFEDLYVY

SEQ ID NO: 9 - protein, 131 aa, Artificial Sequence
  Mouse VSTM4 Ig-like domain (AA24-AA154)
LNVTVSPGPVVDYLEGENATLLCHVSQKRRKDSLLAVRWFFAPDGSQEALMVKMTKLRIIQYYGNFSRTANQQRLRLLEERRGVLYRLSVLTLRPTDQGQYVCKVQEISKHRNKWTAWSNGSSATEMRVIS

SEQ ID NO: 10 - protein, 106 aa, Artificial Sequence
  Mouse VSTM4 Ig-like V domain (AA24-AA129)
LNVTVSPGPVVDYLEGENATLLCHVSQKRRKDSLLAVRWFFAPDGSQEALMVKMTKLRIIQYYGNFSRTANQQRLRLLEERRGVLYRLSVLTLRPTDQGQYVCKVQ

SEQ ID NO: 11 - protein, 282 aa, Homo sapiens
  MISC_FEATURE: Human B7-H4 with predicted signal sequence
  MISC_FEATURE (1)..(24): Predicted signal sequence
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 12 - protein, 259 aa, Artificial Sequence
  Human B7-H4 ECD (AA1-AA259) with predicted signal sequence
  MISC_FEATURE (1)..(24): Predicted signal sequence
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKAS

SEQ ID NO: 13 - protein, 235 aa, Artificial Sequence
  Human B7-H4 ECD (AA25-AA259) without predicted signal sequence
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKAS

SEQ ID NO: 14 - protein, 112 aa, Artificial Sequence
  Human B7-H4 Ig-like V domain 1 (AA35-AA146)
HSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYK

SEQ ID NO: 15 - protein, 89 aa, Artificial Sequence
  Human B7-H4 Ig-like V domain 2 (AA153-AA241)
PEVNVDYNASSETLRCEAPRWFPQPTVVWASQVDQGANFSEVSNTSFELNSENVTMKVVSVLYNVTINNTYSCMIENDIAKATGDIKVT

SEQ ID NO: 16 - protein, 283 aa, Mus musculus
  MISC_FEATURE: Mouse B7-H4 with predicted signal sequence
  MISC_FEATURE (1)..(24): Predicted signal sequence
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVFSSAFVAGWALLSLSCCLMLR

SEQ ID NO: 17 - protein, 257 aa, Artificial Sequence
  Mouse B7-H4 ECD (AA1-AA257) with predicted signal sequence
  MISC_FEATURE (1)..(24): Predicted signal sequence
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSG

SEQ ID NO: 18 - protein, 233 aa, Artificial Sequence
  Mouse B7-H4 ECD (AA25-AA257) without predicted signal sequence
LIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSG

SEQ ID NO: 19 - protein, 111 aa, Artificial Sequence
  Mouse B7-H4 Ig-like V-type domain 1 (AA35-AA144)
KHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLE

SEQ ID NO: 20 - protein, 69 aa, Artificial Sequence
  Mouse B7-H4 Ig-like V-type domain 2 (AA153-AA241)
WFPQPTVAWASQVDQGANFSEVSNTSFELNSENVTMKVVSVLYNVTINNTYSCMIENDIAKATGDIKVT

SEQ ID NO: 21 - protein, 227 aa, Artificial Sequence
  Human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 230 aa, Artificial Sequence
  Human IgG1 Fc region
KSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 232 aa, Artificial Sequence
  Human IgG1 Fc region
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 224 aa, Artificial Sequence
  Human IgG2 Fc region
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 222 aa, Artificial Sequence
  Mouse IgG1 Fc region
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 26 - protein, 227 aa, Artificial Sequence
  Mouse IgG2a Fc region
IKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - protein, 330 aa, Artificial Sequence [sequence omitted]

SEQ ID NO: 28 - protein, 326 aa, Artificial Sequence
  Human IgG2 Heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 377 aa, Artificial Sequence
  Human IgG3 Heavy chain constant region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 30 - protein, 327 aa, Artificial Sequence
  Human IgG4 Heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 8 aa, Artificial Sequence
  FLAG Tag
DYKDDDDK

SEQ ID NO: 32 - protein, 8 aa, Artificial Sequence
  Linker
ESGGGGVT

SEQ ID NO: 33 - protein, 9 aa, Artificial Sequence
  Linker
LESGGGGVT

SEQ ID NO: 34 - protein, 6 aa, Artificial Sequence
  Linker
GRAQVT

SEQ ID NO: 35 - protein, 6 aa, Artificial Sequence
  Linker
WRAQVT

SEQ ID NO: 36 - protein, 8 aa, Artificial Sequence
  Linker
ARGRAQVT

SEQ ID NO: 37 - protein, 9 aa, Artificial Sequence
  ITAM
  misc_feature (2)..(3): Xaa can be any naturally occurring amino acid
  MISC_FEATURE (4)..(4): Xaa can be either Leu or Ile
  MISC_FEATURE (5)..(5): Xaa can be any naturally occurring amino acid and is repeated 6-8 times
  misc_feature (7)..(8): Xaa can be any naturally occurring amino acid
  MISC_FEATURE (9)..(9): Xaa can be either Leu or Ile
YXXXXYXXX

SEQ ID NO: 38 - protein, 7 aa, Artificial Sequence
  ITIM
  MISC_FEATURE (1)..(1): Xaa is Ser, Ile, Val, or Leu
  misc_feature (3)..(3): Xaa can be any naturally occurring amino acid
  misc_feature (5)..(6): Xaa can be any naturally occurring amino acid
  MISC_FEATURE (7)..(7): Xaa can be Ile, Val, or Leu
XLXYXXX

SEQ ID NO: 39 - protein, 6 aa, Artificial Sequence
  ITSM
  misc_feature (2)..(2): Xaa can be any naturally occurring amino acid
  misc_feature (4)..(5): Xaa can be any naturally occurring amino acid
  MISC_FEATURE (6)..(6): Xaa is Val or Ile
TXYXXX

(19) United States
(12) Patent Application Publication
(10) Pub. No.: US 2017/0267758 A1
(43) Pub. Date: Sep. 21, 2017

(54) IMMUNOTHERAPY WITH BINDING AGENTS

(71) Applicant: ONCOMED PHARMACEUTICALS, INC., Redwood City, CA (US)

(72) Inventors: Austin L. GURNEY, San Francisco, CA (US); Ming-hong XIE, Foster City, CA (US); James Gordon PORTER, Newark, CA (US)

(73) Assignee: ONCOMED PHARMACEUTICALS, INC., Redwood City, CA (US)

(21) Appl. No.: 15/310,132

(22) PCT Filed: May 13, 2015
(86) PCT No.: PCT/US15/30472

§ 371 Date: Nov. 10, 2016

Related U.S. Application Data
(60) Provisional application No. 61/992,456, filed on May 13, 2014.

(51) Int. Cl. C07K 16/28; C07K 16/30; C07K 14/705

(52) U.S. Cl. CPC C07K 16/2803; C07K 14/70503; C07K 16/30; C07K 16/2818; C07K 2319/30; A61K 38/00

(57) ABSTRACT

Binding agents that modulate the immune response are disclosed. The binding agents may include antibodies, soluble receptors, and/or polypeptides. Also disclosed are methods of using the binding agents for the treatment of diseases such as cancer.

### CROSS-REFERENCE TO RELATED APPLICATIONS

This application claims priority benefit of U.S. Provisional Application No. 61/992,456, filed May 13, 2014, which is hereby incorporated by reference herein in its entirety.

### FIELD OF THE INVENTION

This invention generally relates to agents that modulate the immune response, such as antibodies, soluble receptors, and small molecules, as well as to methods of using the agents for the treatment of diseases such as cancer.

### BACKGROUND OF THE INVENTION

The basis for immunotherapy is the manipulation and/or modulation of the immune system, including both innate immune responses and adaptive immune responses. The general aim of immunotherapy is to treat diseases by controlling the immune response to a “foreign agent”, for example a pathogen or a tumor cell. However, in some instances immunotherapy is used to treat autoimmune diseases which may arise from an abnormal immune response against proteins, molecules, and/or tissues normally present in the body Immunotherapy may include methods to induce or enhance specific immune responses or to inhibit or reduce specific immune responses. The immune system is a highly complex system made up of a great number of cell types, including but not limited to, T-cells, B-cells, natural killer cells, antigen-presenting cells, dendritic cells, monocytes, and macrophages. These cells possess complex and subtle systems for controlling their interactions and responses. The cells utilize both activating and inhibitory mechanisms and feedback loops to keep responses in check and not allow negative consequences of an uncontrolled immune response (e.g., autoimmune diseases).

Generally, an immune response is initiated through antigen recognition by the T-cell receptor (TCR) and is regulated by a balance between stimulatory and inhibitory signals (i e, immune checkpoints). Under normal conditions, immune checkpoints are necessary to maintain a balance between activating and inhibitory signals and to ensure the development of an effective immune response while safeguarding against the development of autoimmunity or damage to tissues when the immune system is responding to a foreign or pathogenic agent. An important immune checkpoint receptor is CTLA4 which is expressed on T-cells and is highly expressed on regulatory T-cells (Tregs). CTLA4 is considered to act as an inhibitory molecule or an immune response “brake” and primarily regulates the amplitude of T-cell activation. CTLA4 counteracts the activity of the co-stimulatory receptor, CD28, which acts in concert with the TCR to activate T-cells. CTLA4 and CD28 share identical ligands or counter-receptors, B7-1 (CD80) and B7-2 (CD86) and the balance of the immune response probably involves competition of CTLA4 and CD28 for binding to the ligands. Another important immune checkpoint receptor is PD1 which is expressed on T-cells after activation, highly expressed on Tregs, and expressed on other activated cells including B-cells and natural killer (NK) cells. Similar to CTLA4, PD1 is considered to act as an inhibitory molecule and brake on the immune response. There are two ligands/counter-receptors for PD1, PDL1 (also known as B7-H1 and CD247) and PDL2 (also known as B7-DC and CD273). (See, Pardoll, 2012, Nature Reviews Cancer, 12:252-264).

The concept of cancer immunosurveillance is based on the theory that the immune system can recognize tumor cells, mount an immune response, and suppress the development and/or progression of a tumor. However, it is clear that many cancerous cells have developed mechanisms to evade the immune system which can allow for uninhibited growth of tumors Immune checkpoints can be dysregulated by tumors and may be manipulated by tumors to be used as an immune resistance mechanism. Cancer immunotherapy focuses on the development of agents that can activate and/or boost the immune system to achieve a more effective response to killing tumor cells and inhibiting tumor growth.

### BRIEF SUMMARY OF THE INVENTION

The present invention provides agents, such as antibodies, soluble receptors, and small molecules that modulate the immune response. In some embodiments, the agents activate or increase the immune response to cancer and/or a tumor. The invention also provides compositions, such as pharmaceutical compositions, comprising the agents. The invention further provides methods of administering the agents to a subject in need thereof. In some embodiments, the invention provides methods of using the agents for cancer immunotherapy.

In one aspect, the present invention provides agents that bind V-set and transmembrane domain-containing protein 4 (VSTM4). In some embodiments, the invention provides an agent that specifically binds the extracellular domain, or a fragment thereof, of VSTM4. In some embodiments, the agent specifically binds the Ig-like domain of VSTM4. In some embodiments, the agent specifically binds the IgV domain of VSTM4. In some embodiments, the agent specifically binds human VSTM4. In some embodiments, the agent specifically binds mouse VSTM4. In some embodiments, the agent specifically binds human VSTM4 and mouse VSTM4. In some embodiments, the agent binds within amino acids 24-180 of human VSTM4 and/or mouse VSTM4. In some embodiments, the agent binds within amino acids 24-155 of human VSTM4 and/or mouse VSTM4. In some embodiments, the agent binds with amino acids 24-130 of human VSTM4 and/or mouse VSTM4. In some embodiments, the agent binds within SEQ ID NO:3, SEQ ID NO:4, SEQ ID NO:5, SEQ ID NO:8, SEQ ID NO:9, SEQ ID NO:10, or a fragment thereof.

As used herein, an “agent” or “binding agent” includes but is not limited to, an antibody, a soluble receptor, a secreted (e.g., soluble) protein, a polypeptide, and a small molecule.

In some embodiments, the agent is an antibody. In some embodiments, the antibody is a monoclonal antibody, a chimeric antibody, a humanized antibody, a human antibody, a bispecific antibody, or an antibody fragment.

In some embodiments, the agent is a soluble receptor or a soluble protein. In some embodiments, the soluble receptor comprises the extracellular domain, or a fragment thereof, of VSTM4. In some embodiments, the soluble receptor comprises the extracellular domain, or a fragment thereof, of human VSTM4. In some embodiments, the soluble receptor comprises the extracellular domain, or a fragment thereof, of mouse VSTM4. In some embodiments, the soluble receptor comprises the Ig-like domain of VSTM4. In some embodiments, the soluble receptor comprises the IgV domain of VSTM4. In some embodiments, the soluble receptor comprises amino acids 24-180 of human VSTM4 or amino acids 24-179 of mouse VSTM4. In some embodiments, the soluble receptor comprises amino acids 24-155 of human VSTM4 or amino acids 24-154 of mouse VSTM4. In some embodiments, the soluble receptor comprises amino acids 24-130 of human VSTM4 or amino acids 24-129 of mouse VSTM4. In some embodiments, the soluble receptor comprises SEQ ID NO:3, SEQ ID NO:4, SEQ ID NO:5, SEQ ID NO:8, SEQ ID NO:9, SEQ ID NO:10, or a fragment thereof. In some embodiments, the soluble receptor or soluble protein is a fusion protein. In some embodiments, the fusion protein comprises a non-VSTM4 polypeptide (i.e., a heterologous protein). In some embodiments, the fusion protein comprises a Fc region. In some embodiments, the non-VSTM4 polypeptide comprises a Fc region. In some embodiments, the Fc region is selected from the group consisting of SEQ ID NO:21, SEQ ID NO:22, SEQ ID NO:23, SEQ ID NO:24, SEQ ID NO:25, and SEQ ID NO:26.

In some embodiments of each of the aforementioned aspects and embodiments, as well as other aspects and embodiments described herein, the agent is monovalent. In some embodiments, the agent is bivalent. In some embodiments, the agent is monospecific. In some embodiments, the agent is bispecific.

In some embodiments of each of the aforementioned aspects and embodiments, as well as other aspects and embodiments described herein, the agent is a heteromultimeric protein. In some embodiments, the agent is a heterodimeric protein. In some embodiments, the heterodimeric protein comprises a first polypeptide which binds VSTM4 and a second polypeptide which binds a second target. In some embodiments, the heterodimeric protein comprises a first polypeptide which binds VSTM4 and a second polypeptide which is an immune response stimulating agent. In some embodiments, the heterodimeric protein comprises a first polypeptide which binds B7-H4 and a second polypeptide which binds a second target. In some embodiments, the heterodimeric protein comprises a first polypeptide which binds B7-H4 and a second polypeptide which is an immune response stimulating agent. In some embodiments, the heterodimeric protein comprises a first polypeptide comprising an agent described herein and a second polypeptide comprising an immune response stimulating agent. In some embodiments, the immune response stimulating agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1), interleukin 2 (IL-2), B7-1 (CD80), B7-2 (CD86), anti-CD3 antibody, anti-CTLA4 antibody, anti-PD-L1 antibody, and anti-PD1 antibody. In some embodiments, the heterodimeric protein comprises two polypeptides, wherein each polypeptide comprises a human IgG2 CH3 domain, and wherein the amino acids at positions corresponding to positions 249 and 288 of SEQ ID NO:28 of one IgG2 CH3 domain are replaced with glutamate or aspartate, and wherein the amino acids at positions corresponding to positions 236 and 278 of SEQ ID NO:28 of the other IgG2 CH3 domain are replaced with lysine.

In some embodiments of each of the aforementioned aspects and embodiments, as well as other aspects and embodiments described herein, the binding agent induces, activates, promotes, increases, enhances, and/or prolongs an immune response. In some embodiments, the binding agent increases cell-mediated immunity. In some embodiments, the binding agent increases T-cell activity. In some embodiments, the agent increases cytolytic T-cell (CTL) activity. In some embodiments, the agent increases natural killer (NK) cell activity. In some embodiments, the agent decreases Treg activity. In some embodiments, the agent is an antagonist of B7-H4-mediated signaling. In some embodiments, the agent is an antagonist of VSTM4 signaling. In some embodiments, the agent inhibits VSTM4 signaling. In some embodiments, the agent inhibits or blocks the interaction between B7-H4 and VSTM4.

In some embodiments of each of the aforementioned aspects and embodiments, as well as other aspects and embodiments described herein, the agent specifically binds VSTM4 and disrupts signaling of a B7-H4/VSTM4 pathway. In some embodiments, the agent specifically binds B7-H4 and disrupts signaling of a B7-H4 pathway. In some embodiments, the agent specifically binds VSTM4 and the agent disrupts binding of VSTM4 to B7-H4, and/or disrupts B7-H4 activation of VSTM4 signaling. In some embodiments, the agent specifically binds B7-H4 and the agent disrupts binding of B7-H4 to VSTM4, and/or disrupts B7-H4 activation of VSTM4 signaling. In some embodiments, the agent disrupts binding of B7-H4 to VSTM4. In some embodiments, the agent disrupts binding of VSTM4 to B7-H4. In some embodiments, the agent disrupts B7-H4 activation of VSTM4 signaling. In some embodiments, the agent inhibits activity of VSTM4. In some embodiments, the agent inhibits the inhibitory activity of VSTM4. In some embodiments, the agent induces, activates, promotes, increases, enhances, and/or prolongs an immune response.

In some embodiments of each of the aforementioned aspects and embodiments, as well as other aspects and embodiments described herein, the agent specifically binds B7-H4 and the agent disrupts the interaction between B7-H4 and VSTM4, and/or disrupts B7-H4 activation of VSTM4 signaling. In some embodiments, the agent disrupts the interaction between B7-H4 and VSTM4. In some embodiments, the agent disrupts the interaction between VSTM4 and B7-H4. In some embodiments, the agent disrupts B7-H4 activation of VSTM4 signaling. In some embodiments, the agent induces, activates, promotes, increases, enhances, or prolongs an immune response.

In another aspect, the invention provides compositions comprising an antibody, a soluble receptor, a soluble protein, a polypeptide, or a binding agent described herein. Methods of using a composition comprising an agent described herein are also provided.

In another aspect, the invention provides pharmaceutical compositions comprising an antibody, a soluble receptor, a soluble protein, a polypeptide, or a binding agent described herein and a pharmaceutically acceptable carrier. Methods of treating cancer and/or inhibiting tumor growth in a subject (e.g., a human) comprising administering to the subject an effective amount of a composition comprising an agent described herein are also provided. Methods of treating a viral infection in a subject (e.g., a human) comprising administering to the subject an effective amount of a composition comprising an agent described herein are also provided.

In certain embodiments of each of the aforementioned aspects, as well as other aspects and/or embodiments described elsewhere herein, the antibody, the soluble receptor, the soluble protein, the polypeptide, or the binding agent is isolated. In certain embodiments, the antibody, the soluble receptor, the soluble protein, the polypeptide, or the binding agent is substantially pure.

In another aspect, the invention provides polynucleotides comprising a polynucleotide that encodes an antibody, a soluble receptor, a soluble protein, a polypeptide, or a binding agent described herein. In some embodiments, the polynucleotide is isolated. In some embodiments, the invention provides vectors that comprise the polynucleotides, as well as cells that comprise the vectors and/or the polynucleotides. In some embodiments, the invention also provides cells comprising or producing an antibody, a soluble receptor, a soluble protein, a polypeptide, or a binding agent described herein. In some embodiments, the cell is a monoclonal cell line.

In another aspect, the invention provides methods of modulating the immune response of a subject. In some embodiments, the invention provides a method of inducing an immune response in a subject comprising using an agent described herein. In some embodiments, the invention provides a method of activating an immune response in a subject comprising using an agent described herein. In some embodiments, the invention provides a method of promoting an immune response in a subject comprising using an agent described herein. In some embodiments, the invention provides a method of increasing an immune response in a subject comprising using an agent described herein. In some embodiments, the invention provides a method of enhancing an immune response in a subject comprising using an agent described herein. In some embodiments, the invention provides a method of prolonging an immune response in a subject comprising using an agent described herein. In some embodiments, the immune response is to an antigenic stimulation. In some embodiments, the antigenic stimulation is a tumor or a tumor cell. In some embodiments, the antigenic stimulation is a pathogen. In some embodiments, the antigenic stimulation is a virus. In some embodiments, the antigenic stimulation is a virally-infected cell. In some embodiments, the invention provides a method of increasing the activity of immune cells. In some embodiments, the invention provides a method of increasing the activity of immune cells comprising contacting the cells with an effective amount of an agent described herein. In some embodiments, the immune cells are T-cells, NK cells, monocytes, macrophages, and/or B-cells. In some embodiments, the invention provides a method of increasing the activity of NK cells in a subject comprising administering to the subject a therapeutically effective amount of an agent described herein. In some embodiments, the invention provides a method of increasing the activity of T-cells in a subject comprising administering to the subject a therapeutically effective amount of an agent described herein. In some embodiments, the invention provides a method of increasing the activation of T-cells and/or NK cells in a subject comprising administering to the subject a therapeutically effective amount of an agent described herein. In some embodiments, the invention provides a method of increasing the T-cell response in a subject comprising administering to the subject a therapeutically effective amount of an agent described herein. In some embodiments, the invention provides a method of increasing the activity of CTLs in a subject comprising administering to the subject a therapeutically effective amount of an agent described herein.

In another aspect, the invention provides methods of inducing, activating, promoting, increasing, enhancing, or prolonging an immune response in a subject, comprising administering to the subject a therapeutically effective amount of an agent described herein. In some embodiments, the invention provides methods of inducing, activating, promoting, increasing, enhancing, or prolonging an immune response in a subject, comprising administering to the subject a therapeutically effective amount of an agent that disrupts the signaling of a B7-H4/VSTM4 pathway. In some embodiments, the disruption of the signaling of a B7-H4/VSTM4 pathway inhibits the B7-H4-mediated suppression of immune responses. In some embodiments, the immune response is against a tumor or cancer. In some embodiments, the immune response is against a viral infection, a viral antigen, or a virally-infected cell.

In another aspect, the invention provides methods of inhibiting tumor growth comprising contacting a tumor or tumor cell with an effective amount of an agent described herein. In some embodiments, a method of inhibiting growth of a tumor comprises contacting a tumor or tumor cell with an effective amount of an agent that disrupts the interaction between B7-H4 and VSTM4.

In another aspect, the invention provides methods of inhibiting tumor growth in a subject comprising administering to the subject a therapeutically effective amount of an agent described herein. In some embodiments, a method of inhibiting growth of a tumor in a subject comprises administering to the subject a therapeutically effective amount of an agent that disrupts the interaction between B7-H4 and VSTM4.

In another aspect, the invention provides methods of treating cancer in a subject comprising administering to the subject a therapeutically effective amount of an agent described herein. In some embodiments, a method of treating cancer in a subject comprises administering to the subject a therapeutically effective amount of an agent that disrupts the interaction between B7-H4 and VSTM4.

In another aspect, the invention provides methods of stimulating a protective response in a subject comprising administering to the subject a therapeutically effective amount of an agent described herein in combination with an antigen of interest. In some embodiments, the antigen of interest is a tumor antigen. In some embodiments, the antigen of interest is a cancer stem cell marker.

In some embodiments of each of the aforementioned aspects and embodiments, as well as other aspects and embodiments described herein, the methods comprise administering to the subject an immune response stimulating agent. In some embodiments, the immune response stimulating agent is selected from the group consisting of GM-CSF, M-CSF, G-CSF, IL-3, IL-12, IL-1, IL-2, B7-1 (CD80), B7-2 (CD86), anti-CD3 antibodies, anti-CTLA-4 antibodies, anti-CD28 antibodies, anti-PD-L1 antibodies, and anti-PD1 antibodies.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE FIGURES

FIG. 1. Family tree of B7 family members.

FIG. 2. FACS analysis of binding interactions between B7-H4 and VSTM4. HEK-293T cells were transiently transfected with a cDNA expression vector encoding membrane-bound VSTM4-CD4TM-GFP and then subsequently mixed with soluble B7-H4-COMP-FLAG. Specific binding is indicated by the presence of signal within the dark square overlay on each FACS plot.

FIG. 3. Sequence of human VSTM4 (SEQ ID NO:1). The predicted signal sequence is underlined (solid line); the predicted Ig-like domain is underlined (dotted line); and the predicted transmembrane domain is boxed. The ITIM and ITSM sequences are marked.

FIG. 4. Diagrammatic representation of immune checkpoint signaling systems.

### DETAILED DESCRIPTION OF THE INVENTION

The present invention provides novel agents, including, but not limited to, antibodies, polypeptides, soluble receptors, and soluble proteins that modulate the immune response. The agents include agonists and antagonists of receptors and counter-receptors (or ligands) that are members of the immunoglobulin superfamily involved in cell interactions and immune response signaling. Related polypeptides and polynucleotides, compositions comprising the agents, pharmaceutical compositions comprising the agents, and methods of making the agents are also provided. Methods of screening for agents that modulate the immune response are provided. Methods of using the novel agents, such as methods of activating an immune response, methods of inducing an immune response, methods of promoting an immune response, methods of increasing an immune response, methods of prolonging an immune response, methods of activating natural killer (NK) cells and/or T-cells, methods of increasing the activity of NK cells and/or T-cells, methods of promoting the activity of NK cells and/or T-cells, methods of decreasing the activity of Treg cells, methods of inhibiting tumor growth, and/or methods of treating cancer are further provided.

The CD28/CTLA4 signaling system is recognized as containing two ligands or counter-receptors, B7-1 (CD80) and B7-2 (CD86) which each bind to CTLA4 and CD28. Within this signaling axis, CD28 serves as an activating receptor, whereas CTLA4 serves as an inhibitory receptor. The intracellular domain of CD28 contains an immunoreceptor tyrosine-based activation motif or ITAM characterized by the consensus sequence YXX(L/I)(X)₍₆₋₈₎YXX(L/I) (SEQ ID NO:37), that is, at least in part, responsible for the stimulatory activity of CD28. In comparison, the intracellular domain of CTLA4 contains an immunoreceptor tyrosine-based inhibitory motif or ITIM characterized by the consensus sequence (S/I/V/L)XYXX(I/V/L) (SEQ ID NO:38), that is, at least in part, responsible for the inhibitory activity of CTLA4 (Barrow A et al., 2006, Eur J Immunol., 36:1646-53). In each motif consensus sequence the “X” represents any amino acid.

The PD-1 signaling system is recognized as containing two ligands or counter-receptors, PD-L1 (B7-H2) and PD-L2 (B7-DC), which each bind to the PD-1 receptor. Similar to CTLA4, PD-1 contains an ITIM which is responsible for providing an inhibitory signal to T-cells. It is noteworthy that there has been no activating receptor identified for PD-L1 or PD-L2 that would correspond in an analogous fashion to the CD28 receptor utilized by B7-1 and B7-2. However, there has been speculation that such a receptor or receptors exist (see, e.g., Ishiwata et al., 2010, J. Immunol., 184:2086-2094; Shin et al., 2003, J. Exp. Med., 198:31-38; Shin et al., 2005, J. Exp. Med., 201:1531-1541; Wang et al., 2003, J. Exp. Med., 197:1083-1091).

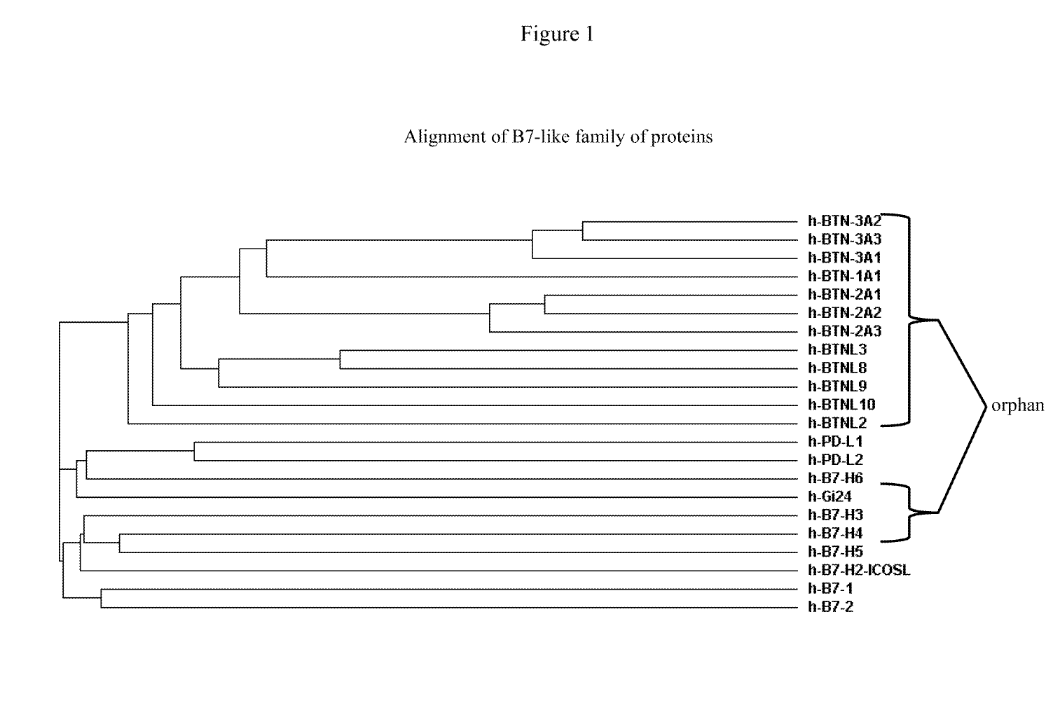

B7-H4 is a member of the B7 family, which includes, but may not be limited to, B7-1, B7-2, PD-L1, PD-L2, B7-H2, B7-H3, B7-H4, B7-H5, B7-H6, Gi24, and the butyrophilin and butyrophilin-like proteins. A family tree depicting members of the B7 family is shown in FIG. 1. A number of B7 proteins are considered to be “orphan molecules” as their interacting receptors, either stimulatory or inhibitory, have not yet been identified and/or reported. This group includes PD-L1 (stimulatory receptor), PD-L2 (stimulatory receptor), B7-H3, B7-H4, B7-H5, Gi24, and the BTN family of proteins. B7-H4 has been shown to not bind known CD28 family members such as CD28, CTLA-4, ICOS, and PD-1 (Sica et al., 2003, Immunity, 18:849-861). Functional studies using B7-H4 transfectants and B7-H4-Ig fusion proteins demonstrate that B7-H4 delivers a signal that inhibits T-cell proliferation, cell-cycle progression, and IL-2 production. It is believed that B7-H4 may be part of another important immune checkpoint signaling pathway. Thus it would be highly advantageous to identify the receptor for B7-H4 and to develop agents that inhibit the B7-H4 signaling pathway.

### I. Definitions

To facilitate an understanding of the present invention, a number of terms and phrases are defined below.

The terms “agonist” and “agonistic” as used herein refer to or describe an agent that is capable of, directly or indirectly, substantially inducing, activating, promoting, increasing, or enhancing the biological activity of a target and/or a pathway. The term “agonist” is used herein to include any agent that partially or fully induces, activates, promotes, increases, or enhances the activity of a protein. Suitable agonists specifically include, but are not limited to, agonist antibodies or fragments thereof, soluble receptors, other fusion proteins, polypeptides, and small molecules.

The terms “antagonist” and “antagonistic” as used herein refer to or describe an agent that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein. Suitable antagonist agents specifically include, but are not limited to, antagonist antibodies or fragments thereof, soluble receptors, other fusion proteins, polypeptides, and small molecules.

The terms “modulation” and “modulate” as used herein refer to a change or an alteration in a biological activity. Modulation includes, but is not limited to, stimulating or inhibiting an activity. Modulation may be an increase or a decrease in activity, a change in binding characteristics, or any other change in the biological, functional, or immunological properties associated with the activity of a protein, a pathway, a system, or other biological targets of interest.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies, multispecific antibodies such as bispecific antibodies generated from at least two intact antibodies, bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site.

The term “variable region” of an antibody refers to the variable region of the antibody light chain, or the variable region of the antibody heavy chain, either alone or in combination. The variable regions of the heavy and light chain each consist of four framework regions (FR) connected by three complementarity determining regions (CDRs), also known as “hypervariable regions”. Generally, the CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen recognition site (antigen-binding site). Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability (Jones et al., 1986, Nature, 321:522-525; Riechmann et al., 1988, Nature, 332:323-327; Verhoeyen et al., 1988, Science, 239:1534-1536). In some instances, the Fv framework region residues of a human immunoglobulin are replaced with the corresponding residues in an antibody from a non-human species that has the desired specificity, affinity, and/or binding capability. The humanized antibody can be further modified by the substitution of additional residues either in the Fv framework region and/or within the replaced non-human residues to refine and optimize antibody specificity, affinity, and/or binding capability. The humanized antibody may comprise substantially all of at least one, and typically two, variable domains containing all or substantially all of the CDRs that correspond to the non-human immunoglobulin whereas all or substantially all of the framework regions are those of a human immunoglobulin sequence. The humanized antibody can also comprise at least a portion of an immunoglobulin constant region or domain (Fc), typically that of a human immunoglobulin.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human) to avoid eliciting an immune response in that species.

The phrase “affinity-matured antibody” as used herein refers to an antibody with one or more alterations in one or more CDRs that result in an improvement in the affinity of the antibody for antigen as compared to a parent antibody that does not possess those alterations(s). Preferred affinity-matured antibodies will have nanomolar or even picomolar affinities for the target antigen. Affinity-matured antibodies are produced by procedures known in the art, for example, affinity maturation by heavy chain variable region and light chain variable region domain shuffling, random mutagenesis of CDR and/or framework residues, or site-directed mutagenesis of CDR and/or framework residues.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

As used herein, the term “soluble receptor” refers to an extracellular fragment of a receptor protein that can be secreted from a cell in soluble form. The term “soluble receptor” encompasses a molecule comprising the entire extracellular domain, or a portion of the extracellular domain. As used herein, the term “soluble protein” refers to a protein or a fragment thereof that can be secreted from a cell in soluble form.

As used herein, the term “linker” or “linker region” refers to a linker inserted between a first polypeptide (e.g., a VSTM4 ECD) and a second polypeptide (e.g., a Fc region). In some embodiments, the linker is a peptide linker. Linkers should not adversely affect the expression, secretion, or bioactivity of the polypeptides. Preferably, linkers are not antigenic and do not elicit an immune response.

The terms “selectively binds” or “specifically binds” mean that an agent reacts or associates more frequently, more rapidly, with greater duration, with greater affinity, or with some combination of the above to the epitope, protein, or target molecule than with alternative substances, including related and unrelated proteins. In certain embodiments “specifically binds” means, for instance, that an agent binds a protein or target with a K_{D} of about 0.1 mM or less, but more usually less than about 1 μM. In certain embodiments, “specifically binds” means that an agent binds a target with a K_{D} of at least about 0.1 μM or less, at least about 0.01 μM or less, or at least about 1 nM or less. Because of the sequence identity between homologous proteins in different species, specific binding can include an agent that recognizes a protein or target in more than one species (e.g., human VSTM4 and mouse VSTM4). Likewise, because of homology within certain regions of polypeptide sequences of different proteins, specific binding can include an agent that recognizes more than one protein or target. It is understood that, in certain embodiments, an agent that specifically binds a first target may or may not specifically bind a second target. As such, “specific binding” does not necessarily require (although it can include) exclusive binding, i.e. binding to a single target. Thus, an agent may, in certain embodiments, specifically bind more than one target. In certain embodiments, multiple targets may be bound by the same antigen-binding site on the agent. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or other members of the immunoglobulin superfamily, in certain embodiments, the polypeptides can occur as single chains or as associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble receptors, and/or antibodies of the invention do not abrogate the binding of the polypeptide, soluble receptor, or antibody containing the amino acid sequence, to the target binding site. Methods of identifying nucleotide and amino acid conservative substitutions which do not eliminate binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, soluble receptor, antibody, polynucleotide, vector, cell, or composition which is “isolated” is a polypeptide, soluble receptor, antibody, polynucleotide, vector, cell, or composition which is in a form not found in nature. Isolated polypeptides, soluble receptors, antibodies, polynucleotides, vectors, cells, or compositions include those which have been purified to a degree that they are no longer in a form in which they are found in nature. In some embodiments, a polypeptide, soluble receptor, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “immune response” as used herein includes responses from both the innate immune system and the adaptive immune system. It includes both T-cell and B-cell responses (e.g., cell-mediated and/or humoral immune responses), as well as responses from other cells of the immune system such as natural killer (NK) cells, monocytes, macrophages, etc.

The terms “cancer” and “cancerous” as used herein refer to or describe the physiological condition in mammals in which a population of cells are characterized by unregulated cell growth. “Cancer” as used herein refers to solid cancers/tumors and hematologic cancers.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an appropriate host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into appropriate hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to a substance approved or approvable by a regulatory agency of the Federal or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one binding agent (e.g., an antibody) of the present disclosure, and which does not destroy the pharmacological activity thereof and is nontoxic when administered in doses sufficient to deliver a therapeutic effect.

The terms “effective amount” or “therapeutically effective amount” or “therapeutic effect” refer to an amount of an agent, an antibody, a soluble receptor, polypeptide, polynucleotide, small organic molecule, or other drug effective to “treat” a disease or disorder in a subject such as, a mammal. In the case of cancer or a tumor, the therapeutically effective amount of an agent (e.g., antibody, soluble receptor, or soluble protein) has a therapeutic effect and as such can boost the immune response, boost the anti-tumor response, increase cytolytic activity of immune cells, increase killing of tumor cells by immune cells, reduce the number of tumor cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods of the present invention if the patient shows one or more of the following: an increased immune response, an increased anti-tumor response, increased cytolytic activity of immune cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Binding Agents

The proteins in the B7 family are cell surface-anchored proteins generally characterized by an N-terminal immunoglobulin variable domain-like region (IgV) followed by at least one immunoglobulin constant domain-like region (IgC). B7-1 and B7-2 have been shown to bind or interact with CTLA4 and CD28; PD-L1 and PD-L2 have been shown to bind or interact with PD-1 and at least one unknown receptor. B7-H2 has been shown to bind or interact with ICOS. B7-H6 has been shown to bind or interact with NKp30. The receptor(s) for B7-H3 and B7-H5 are unknown at this point in time. However, for the first time, a receptor for B7-H4 has been identified and shown to be V-set and transmembrane domain-containing protein 4 or VSTM4.

VSTM4, also known as C10orf72, is a 320 amino acid single-pass type I membrane protein that contains one Ig-like domain. The full-length amino acid (aa) sequences of human VSTM4 (UniProt no. Q8IW00) and mouse VSTM4 (UniProt no. T1NXB5) are known in the art and are provided herein as SEQ ID NO:1 and SEQ ID NO:6, respectively. The full-length amino acid sequences of human B7-H4 (UniProt no. Q727D3; also known as V-set domain containing T-cell activation inhibitor 1 or VTCN1) and mouse B7-H4 (UniProt no. Q7TSP5) are known in the art and are provided herein as SEQ ID NO:11 and SEQ ID NO:16, respectively. As used herein, reference to amino acid positions refer to the numbering of full-length amino acid sequences including the signal sequence.

The present invention provides agents that bind VSTM4. The present invention also provides agents that bind B7-H4. In some embodiments, an agent binds VSTM4 and interferes with the interaction of VSTM4 with a second protein. In some embodiments, an agent binds VSTM4 and interferes with the interaction of VSTM4 with B7-H4. In some embodiments, the agent is an antibody that interferes with the interaction of VSTM4 with B7-H4. In some embodiments, the agent is an antibody that specifically binds VSTM4. In some embodiments, the agent comprises an antibody that interferes with the interaction of VSTM4 with B7-H4. In some embodiments, an agent is a soluble receptor that interferes with the interaction of VSTM4 with a second protein. In some embodiments, an agent is a soluble receptor that interferes with the interaction of VSTM4 with B7-H4. In some embodiments, an agent comprises a soluble receptor that interferes with the interaction of VSTM4 with B7-H4. In some embodiments, an agent is a small molecule that interferes with the interaction of VSTM4 with B7-H4. In some embodiments, the small molecule binds VSTM4. In some embodiments, an agent is a small peptide that interferes with the interaction of VSTM4 with B7-H4. In some embodiments, the small peptide binds VSTM4.

In some embodiments, an agent binds B7-H4 and interferes with the interaction of B7-H4 with a second protein. In some embodiments, an agent binds B7-H4 and interferes with the interaction of B7-H4 with VSTM4. In some embodiments, the agent is an antibody that interferes with the interaction of B7-H4 with VSTM4. In some embodiments, the agent is an antibody that specifically binds B7-H4. In some embodiments, the agent comprises an antibody that interferes with the interaction of B7-H4 with VSTM4. In some embodiments, an agent is a soluble receptor that interferes with the interaction of B7-H4 with a second protein. In some embodiments, an agent is a soluble receptor that interferes with the interaction of B7-H4 with VSTM4. In some embodiments, the agent is a soluble receptor that binds B7-H4. In some embodiments, an agent comprises a soluble receptor that interferes with the interaction of B7-H4 with VSTM4. In some embodiments, an agent is a small molecule that interferes with the interaction of B7-H4 with VSTM4. In some embodiments, the small molecule binds B7-H4. In some embodiments, an agent is a small peptide that interferes with the interaction of B7-H4 with VSTM4. In some embodiments, the small peptide binds B7-H4.

In some embodiments, an agent specifically binds VSTM4 and the agent disrupts binding of VSTM4 to B7-H4, and/or disrupts B7-H4 activation of VSTM4 signaling. In some embodiments, an agent specifically binds B7-H4 and the agent disrupts binding of B7-H4 to VSTM4, and/or disrupts B7-H4 activation of VSTM4 signaling. In some embodiments, the agent disrupts binding of VSTM4 to B7-H4. In some embodiments, the agent disrupts binding of B7-H4 to VSTM4. In some embodiments, the agent disrupts B7-H4 activation of VSTM4 signaling. In some embodiments, the agent induces, enhances, augments, increases, or prolongs an immune response.

In some embodiments, an agent specifically binds VSTM4 and the agent disrupts the interaction of VSTM4 with B7-H4, and/or disrupts B7-H4 activation of VSTM4 signaling. In some embodiments, an agent specifically binds B7-H4 and the agent disrupts the interaction of B7-H4 with VSTM4, and/or disrupts B7-H4 activation of VSTM4 signaling. In some embodiments, the agent disrupts the interaction of VSTM4 with B7-H4. In some embodiments, the agent disrupts the interaction of B7-H4 with VSTM4. In some embodiments, the agent disrupts B7-H4 activation of VSTM4 signaling. In some embodiments, the agent induces, enhances, augments, increases, or prolongs an immune response.

In some embodiments, an agent binds VSTM4 with a dissociation constant (K_{D}) of about μM or less, about 100 nM or less, about 40 nM or less, about 20 nM or less, about 10 nM or less, about 1 nM or less, or about 0.1 nM or less. In some embodiments, an agent binds VSTM4 with a K_{D} of about 1 nM or less. In some embodiments, an agent binds VSTM4 with a K_{D} of about 0.1 nM or less. In certain embodiments, an agent described herein binds human VSTM4. In some embodiments, an agent binds a human VSTM4 with a K_{D} of about 0.1 nM or less. In some embodiments, an agent binds both human VSTM4 and mouse VSTM4 with a K_{D} of about 10 nM or less. In some embodiments, an agent binds both human VSTM4 and mouse VSTM4 with a K_{D} of about 1 nM or less. In some embodiments, an agent binds both human VSTM4 and mouse VSTM4 with a K_{D} of about 0.1 nM or less. In some embodiments, the dissociation constant of the agent to VSTM4 is the dissociation constant determined using a VSTM4 fusion protein comprising at least a fragment of VSTM4 immobilized on a Biacore chip.

In some embodiments, an agent binds B7-H4 with a dissociation constant (K_{D}) of about μM or less, about 100 nM or less, about 40 nM or less, about 20 nM or less, about 10 nM or less, about 1 nM or less, or about 0.1 nM or less. In some embodiments, an agent binds B7-H4 with a K_{D} of about 1 nM or less. In some embodiments, an agent binds B7-H4 with a K_{D} of about 0.1 nM or less. In some embodiments, an agent binds human B7-H4 with a K_{D} of about 0.1 nM or less. In some embodiments, an agent binds both human B7-H4 and mouse B7-H4 with a K_{D} of about 10 nM or less. In some embodiments, an agent binds both human B7-H4 and mouse B7-H4 with a K_{D} of about 1 nM or less. In some embodiments, an agent binds both human B7-H4 and mouse B7-H4 with a K_{D} of about 0.1 nM or less. In some embodiments, the dissociation constant of the agent to B7-H4 is the dissociation constant determined using a B7-H4 fusion protein comprising at least a fragment of B7-H4 immobilized on a Biacore chip.

In some embodiments, an agent binds human VSTM4 with a half maximal effective concentration (EC₅₀) of about μM or less, about 100 nM or less, about 40 nM or less, about 20 nM or less, about 10 nM or less, about 1 nM or less, or about 0.1 nM or less.

In some embodiments, an agent binds human B7-H4 with a half maximal effective concentration (EC₅₀) of about μM or less, about 100 nM or less, about 40 nM or less, about 20 nM or less, about 10 nM or less, about 1 nM or less, or about 0.1 nM or less.

In some embodiments, the agent binds VSTM4, i.e., the agent is a “VSTM4-binding agent”. In some embodiments, the agent binds B7-H4, i.e., is a “B7-H4-binding agent”.

In some embodiments, the agent specifically binds the extracellular domain, or a fragment thereof, of VSTM4. In some embodiments, the agent specifically binds the Ig-like domain of VSTM4. In some embodiments, the agent specifically binds the IgV domain of VSTM4. In some embodiments, the agent specifically binds human VSTM4. In some embodiments, the agent specifically binds mouse VSTM4. In some embodiments, the agent specifically binds human VSTM4 and mouse VSTM4. In some embodiments, the agent binds within amino acids 24-180 of human VSTM4 and/or amino acids 24-179 of mouse VSTM4. In some embodiments, the agent binds within amino acids 24-155 of human VSTM4 and/or amino acids 24-154 of mouse VSTM4. In some embodiments, the agent binds with amino acids 24-130 of human VSTM4 and/or amino acids 24-129 of mouse VSTM4. In some embodiments, the agent binds within SEQ ID NO:3, SEQ ID NO:4, SEQ ID NO:5, SEQ ID NO:8, SEQ ID NO:9, SEQ ID NO:10, or a fragment thereof.

In some embodiments, the VSTM4-binding agent is an antibody. In some embodiments, the B7-H4-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent. In some embodiments, the antibody is bivalent. In some embodiments, the antibody is monospecific. In some embodiments, the antibody is bispecific or multispecific. In some embodiments, the antibody is conjugated to a cytotoxic moiety. In some embodiments, the antibody is isolated. In some embodiments, the antibody is substantially pure.

In some embodiments, the VSTM4-binding agents are polyclonal antibodies. In some embodiments, the B7-H4-binding agents are polyclonal antibodies. Polyclonal antibodies can be prepared by any known method. In some embodiments, polyclonal antibodies are raised by immunizing an animal (e.g., a rabbit, rat, mouse, goat, or donkey) by multiple subcutaneous or intraperitoneal injections of the relevant antigen (e.g., a polypeptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, ascites, and the like, of the immunized animal. The polyclonal antibodies can be purified from serum or ascites according to standard methods in the art including, but not limited to, affinity chromatography, ion-exchange chromatography, gel electrophoresis, and dialysis.

In some embodiments, the VSTM4-binding agents are monoclonal antibodies. In some embodiments, the B7-H4-binding agents are monoclonal antibodies. Monoclonal antibodies can be prepared using hybridoma methods known to one of skill in the art. In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a fragment thereof. In some embodiments, the immunizing antigen can be a mouse protein or a fragment thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. In some embodiments, the polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains or the heavy chain and light chain variable regions are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries expressing CDRs and/or variable regions of the desired species.

The polynucleotide(s) encoding a monoclonal antibody can further be modified in a number of different methods using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region(s) can be used to optimize specificity, affinity, etc. of a monoclonal antibody.

In some embodiments, the VSTM4-binding agent is a humanized antibody. In some embodiments, the B7-H4-binding agent is a humanized antibody. Typically, humanized antibodies are human immunoglobulins in which the CDRs are replaced by the CDRs from a non-human species (e.g., mouse, rat, rabbit, hamster, etc.) that have the desired specificity, affinity, and/or binding capability using methods known to one skilled in the art. In some embodiments, the Fv framework region residues of a human immunoglobulin are replaced with the corresponding residues of an antibody from a non-human species. In some embodiments, the humanized antibody can be further modified by the substitution of additional residues either in the Fv framework region and/or within the replaced non-human residues. The humanized antibody may comprise variable domain regions containing all, or substantially all, of the CDRs that correspond to the non-human immunoglobulin whereas all, or substantially all, of the framework regions are those of a human immunoglobulin sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and human anti-mouse antibody (HAMA) responses when administered to a human subject.

In some embodiments, the VSTM4-binding agent is a human antibody. In some embodiments, the B7-H4-binding agent is a human antibody. Human antibodies can be directly prepared using various techniques known in the art. In some embodiments, immortalized human B lymphocytes immunized in vitro or isolated from an immunized individual that produces an antibody directed against a target antigen can be generated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Affinity maturation strategies including, but not limited to, chain shuffling and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production.

This invention also encompasses bispecific antibodies. In some embodiments, a bispecific antibody specifically binds VSTM4 as well as a second antigen. In some embodiments, a bispecific antibody specifically binds B7-H4 as well as a second antigen. Bispecific antibodies are capable of specifically recognizing and binding at least two different epitopes. The different epitopes can either be within the same molecule (e.g., two epitopes on human VSTM4) or on different molecules (e.g., one epitope on VSTM4 and one epitope on a second non-VSTM4 molecule). In some embodiments, the bispecific antibodies are monoclonal human or humanized antibodies. In some embodiments, the antibodies can specifically recognize and bind a first antigen target, (e.g., VSTM4) as well as a second antigen target, such as an effector molecule on a leukocyte (e.g., CD2, CD3, CD28, CD80, or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA.

Techniques for making bispecific antibodies are known by those skilled in the art, see for example, Millstein et al., 1983, Nature, 305:537-539; Brennan et al., 1985, Science, 229:81; Suresh et al., 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; U.S. Pat. No. 5,731,168; and U.S. Patent Publication No. 2011/0123532. Bispecific antibodies can be intact antibodies or antibody fragments. Antibodies with more than two valencies are also contemplated, for example, trispecific antibodies can be prepared. Thus, in certain embodiments the antibodies are multispecific.

In certain embodiments, the VSTM4-binding agent is an antibody fragment. In certain embodiments, the B7-H4-binding agent is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for VSTM4 or derivatives, B7-H4 or derivatives, fragments, analogs, or homologs thereof. In some embodiments, antibody fragments are linear antibody fragments. In certain embodiments, antibody fragments are monospecific or bispecific. In certain embodiments, the VSTM4-binding agent is a scFv. In certain embodiments, the B7-H4-binding agent is a scFv. Various techniques can be used for the production of single-chain antibodies specific for VSTM4 or B7-H4 and are known to those of skill in the art.

It can further be desirable, especially in the case of antibody fragments, to modify an antibody in order to increase (or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., human VSTM4 or B7-H4). In this regard, the variable region may comprise or be derived from any type of mammal that can be induced to mount a humoral response and generate immunoglobulins against the desired target. As such, the variable region of the modified antibodies can be, for example, of human, murine, non-human primate (e.g. cynomolgus monkeys, macaques, etc.), or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived from an antibody of different class and preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the specific activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions, or substitutions of one or more amino acids in one or more domains. The modified antibodies disclosed herein may comprise alterations or modifications to one or more of the three heavy chain constant domains (CH1, CH2 or CH3) and/or to the light chain constant domain. In some embodiments, one or more domains are partially or entirely deleted from the constant regions of the modified antibodies. In some embodiments, the modified antibodies will comprise domain-deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that controls a specific effector function (e.g. complement C1q binding) to be modulated. Such partial deletions of the constant regions may improve selected characteristics of the antibody (e.g., serum half-life) while leaving other desirable functions associated with the constant region intact. Moreover, as alluded to above, the constant regions of the disclosed antibodies may be modified through the mutation or substitution of one or more amino acids that enhances the function of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (when the antibodies are bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using biochemical or molecular engineering techniques well-known to the skilled artisan.

In certain embodiments, a VSTM4-binding agent or B7-H4-binding agent that is an antibody does not have one or more effector functions. For instance, in some embodiments, the antibody has no ADCC activity, and/or no complement-dependent cytotoxicity (CDC) activity. In certain embodiments, the antibody does not bind an Fc receptor and/or complement factors. In certain embodiments, the antibody has no effector function.

The present invention further embraces variants and equivalents which are substantially homologous to the antibodies, or antibody fragments thereof, described herein. In some embodiments, variants may contain conservative substitution mutations, i.e. the substitution of one or more amino acids by similar amino acids. As used herein, conservative substitution refers to the substitution of an amino acid with another within the same general class such as, for example, one acidic amino acid with another acidic amino acid, one basic amino acid with another basic amino acid, or one neutral amino acid by another neutral amino acid.

Thus, the present invention provides methods for producing an antibody that binds VSTM4. In some embodiments, a method produces an antibody that binds human VSTM4. In some embodiments, a method produces an antibody that binds mouse VSTM4. In some embodiments, a method produces an antibody that binds human VSTM4 and mouse VSTM4. In some embodiments, a method for producing an antibody that binds VSTM4 comprises using hybridoma techniques.

In some embodiments, a method for producing an antibody that binds the extracellular domain of human VSTM4 is provided. In some embodiments, the method comprises using the amino acids of SEQ ID NO:1 or a fragment thereof as an immunogen. As used herein, the phrases “a portion thereof” and “a fragment thereof” are used interchangeably. In some embodiments, the method comprises using the amino acids of SEQ ID NO:2 or a fragment thereof as an immunogen. In some embodiments, the method comprises using the amino acids of SEQ ID NO:3 or a fragment thereof as an immunogen. In some embodiments, the method comprises using the amino acids of SEQ ID NO:4 or a fragment thereof as an immunogen. In some embodiments, the method comprises using the amino acids of SEQ ID NO:5 or a fragment thereof as an immunogen.

In some embodiments, a method for producing an antibody that binds the extracellular domain of mouse VSTM4 is provided. In some embodiments, the method comprises using the amino acids of SEQ ID NO:6 or a fragment thereof as an immunogen. In some embodiments, the method comprises using the amino acids of SEQ ID NO:7 or a fragment thereof as an immunogen. In some embodiments, the method comprises using the amino acids of SEQ ID NO:8 or a fragment thereof as an immunogen. In some embodiments, the method comprises using the amino acids of SEQ ID NO:9 or a fragment thereof as an immunogen. In some embodiments, the method comprises using the amino acids of SEQ ID NO:10 or a fragment thereof as an immunogen.

In some embodiments, a method for producing an antibody that binds the extracellular domain of human B7-H4 is provided. In some embodiments, the method comprises using the amino acids of SEQ ID NO:11 or a fragment thereof as an immunogen. In some embodiments, the method comprises using the amino acids of SEQ ID NO:12 or a fragment thereof as an immunogen. In some embodiments, the method comprises using the amino acids of SEQ ID NO:13 or a fragment thereof as an immunogen. In some embodiments, the method comprises using the amino acids of SEQ ID NO:14 or a fragment thereof as an immunogen. In some embodiments, the method comprises using the amino acids of SEQ ID NO:15 or a fragment thereof as an immunogen.

In some embodiments, a method for producing an antibody that binds the extracellular domain of mouse B7-H4 is provided. In some embodiments, the method comprises using the amino acids of SEQ ID NO:16 or a fragment thereof as an immunogen. In some embodiments, the method comprises using the amino acids of SEQ ID NO:17 or a fragment thereof as an immunogen. In some embodiments, the method comprises using the amino acids of SEQ ID NO:18 or a fragment thereof as an immunogen. In some embodiments, the method comprises using the amino acids of SEQ ID NO:19 or a fragment thereof as an immunogen. In some embodiments, the method comprises using the amino acids of SEQ ID NO:20 or a fragment thereof as an immunogen.

In some embodiments, the method of generating an antibody that binds VSTM4 or B7-H4 comprises screening a phage library. In some embodiments, the phage library is a phage library expressing human proteins.

The present invention further provides methods of identifying an antibody that binds VSTM4 or B7-H4. In some embodiments, the antibody is identified by screening using FACS for binding to a protein (e.g., VSTM4) or a fragment thereof. In some embodiments, the antibody is identified by screening using ELISA for binding to a protein (e.g., VSTM4) or a fragment thereof. In some embodiments, the antibody is identified by screening for the effect on cell growth and/or proliferation in an assay. In some embodiments, the antibody is identified by screening for inhibition or blocking of cell growth or proliferation. In some embodiments, the antibody is identified by screening for activation or enhancement of T-cell signaling. In some embodiments, the antibody is identified by screening for inhibition or blocking of T-cell signaling.

In some embodiments, a method of generating an antibody to human VSTM4 comprises immunizing a mammal with a polypeptide comprising the extracellular domain of human VSTM4. In some embodiments, a method of generating an antibody to human VSTM4 comprises immunizing a mammal with a polypeptide comprising at least a fragment of the extracellular domain from VSTM4. In some embodiments, a method of generating an antibody to human VSTM4 comprises immunizing a mammal with a polypeptide comprising at least a fragment of SEQ ID NO:1, SEQ ID NO:2, SEQ ID NO:3, SEQ ID NO:4, or SEQ ID NO:5. In some embodiments, a method of generating an antibody to human VSTM4 comprises immunizing a mammal with a polypeptide comprising at least a fragment of SEQ ID NO:3, SEQ ID NO:4, or SEQ ID NO:5. In some embodiments, the method further comprises isolating antibodies or antibody-producing cells from the mammal.

In some embodiments, a method of generating an antibody to mouse VSTM4 comprises immunizing a mammal with a polypeptide comprising the extracellular domain of mouse VSTM4. In some embodiments, a method of generating an antibody to mouse VSTM4 comprises immunizing a mammal with a polypeptide comprising at least a fragment of the extracellular domain from VSTM4. In some embodiments, a method of generating an antibody to mouse VSTM4 comprises immunizing a mammal with a polypeptide comprising at least a fragment of SEQ ID NO:6, SEQ ID NO:7, SEQ ID NO:8, SEQ ID NO:9, or SEQ ID NO:10. In some embodiments, a method of generating an antibody to mouse VSTM4 comprises immunizing a mammal with a polypeptide comprising at least a fragment of SEQ ID NO:8, SEQ ID NO:9, or SEQ ID NO:10. In some embodiments, the method further comprises isolating antibodies or antibody-producing cells from the mammal.

In some embodiments, a method of generating a monoclonal antibody which binds VSTM4 comprises: (a) immunizing a mammal with a polypeptide comprising at least a fragment of the extracellular domain of VSTM4; (b) isolating antibody-producing cells from the immunized mammal; (c) fusing the antibody-producing cells with cells of a myeloma cell line to form hybridoma cells. In some embodiments, the method further comprises (d) selecting a hybridoma cell expressing an antibody that binds VSTM4. In some embodiments, the antibody binds human VSTM4. In some embodiments, the antibody binds mouse VSTM4. In some embodiments, the antibody binds both human VSTM4 and mouse VSTM4.

In some embodiments, a method of producing an antibody to VSTM4 comprises screening an antibody-expressing library for antibodies that bind VSTM4. In some embodiments, the antibody-expressing library is a phage library. In some embodiments, the antibody-expressing library is a mammalian cell display library. In some embodiments, the screening comprises panning. In some embodiments, the antibody-expressing library is screened using at least a fragment of the extracellular domain of human VSTM4. In some embodiments, antibodies identified in the first screening, are screened again using mouse VSTM4 thereby identifying an antibody that binds human VSTM4 and mouse VSTM4.

In some embodiments, a method of generating an antibody to human B7-H4 comprises immunizing a mammal with a polypeptide comprising the extracellular domain of human B7-H4. In some embodiments, a method of generating an antibody to human B7-H4 comprises immunizing a mammal with a polypeptide comprising at least a fragment of the extracellular domain from B7-H4. In some embodiments, a method of generating an antibody to human B7-H4 comprises immunizing a mammal with a polypeptide comprising at least a fragment of SEQ ID NO:11, SEQ ID NO:12, SEQ ID NO:13, SEQ ID NO:14, or SEQ ID NO:15. In some embodiments, a method of generating an antibody to human B7-H4 protein comprises immunizing a mammal with a polypeptide comprising at least a fragment of SEQ ID NO:13, SEQ ID NO:14, or SEQ ID NO:15. In some embodiments, the method further comprises isolating antibodies or antibody-producing cells from the mammal.

In some embodiments, a method of generating an antibody to mouse B7-H4 comprises immunizing a mammal with a polypeptide comprising the extracellular domain of mouse B7-H4. In some embodiments, a method of generating an antibody to mouse B7-H4 comprises immunizing a mammal with a polypeptide comprising at least a fragment of the extracellular domain from B7-H4. In some embodiments, a method of generating an antibody to mouse B7-H4 comprises immunizing a mammal with a polypeptide comprising at least a fragment of SEQ ID NO:16, SEQ ID NO:17, SEQ ID NO:18, SEQ ID NO:19, or SEQ ID NO:20. In some embodiments, a method of generating an antibody to mouse B7-H4 protein comprises immunizing a mammal with a polypeptide comprising at least a fragment of SEQ ID NO:18, SEQ ID NO:19, or SEQ ID NO:20. In some embodiments, the method further comprises isolating antibodies or antibody-producing cells from the mammal.

In some embodiments, a method of generating a monoclonal antibody which binds human B7-H4 comprises: (a) immunizing a mammal with a polypeptide comprising at least a portion of the extracellular domain from B7-H4; (b) isolating antibody-producing cells from the immunized mammal; (c) fusing the antibody-producing cells with cells of a myeloma cell line to form hybridoma cells. In some embodiments, the method further comprises (d) selecting a hybridoma cell expressing an antibody that binds B7-H4. In some embodiments, the antibody binds human B7-H4. In some embodiments, the antibody binds mouse B7-H4. In some embodiments, the antibody binds both human B7-H4 and mouse B7-H4.

In some embodiments, a method of producing an antibody to human B7-H4 comprises screening an antibody-expressing library for antibodies that bind human B7-H4. In some embodiments, the antibody-expressing library is a phage library. In some embodiments, the antibody-expressing library is a mammalian cell display library. In some embodiments, the screening comprises panning. In some embodiments, the antibody-expressing library is screened using at least a portion of the extracellular domain of human B7-H4. In some embodiments, the antibody-expressing library is screened using at least a fragment of the extracellular domain of human B7-H4. In some embodiments, antibodies identified in the first screening, are screened again using mouse B7-H4 thereby identifying an antibody that binds human B7-H4 and mouse B7-H4.

In certain embodiments, the antibodies described herein are isolated. In certain embodiments, the antibodies described herein are substantially pure.

In certain embodiments, the agent is a soluble receptor. In certain embodiments, a soluble receptor comprises the extracellular domain (ECD) of VSTM4. In some embodiments, a soluble receptor comprises the extracellular domain of B7-H4. In some embodiments, a soluble receptor comprises a fragment of the extracellular domain of VSTM4 (e.g., the IgV domain of VSTM4). In some embodiments, a soluble receptor comprises a fragment of the extracellular domain of B7-H4 (e.g., the IgV domain of B7-H4).

In some embodiments, a soluble receptor comprises the extracellular domain, or a fragment thereof, of human VSTM4. In some embodiments, a soluble receptor comprises the extracellular domain, or a fragment thereof, of mouse VSTM4. In some embodiments, a soluble receptor comprises the Ig-like domain of VSTM4. In some embodiments, a soluble receptor comprises the IgV domain of VSTM4. In some embodiments, a soluble receptor comprises amino acids 24-180 of human VSTM4 or amino acids 24-179 of mouse VSTM4. In some embodiments, a soluble receptor comprises amino acids 24-155 of human VSTM4 or amino acids 24-154 of mouse VSTM4. In some embodiments, a soluble receptor comprises amino acids 24-130 of human VSTM4 or amino acids 24-129 of mouse VSTM4. In some embodiments, a soluble receptor comprises SEQ ID NO:3, SEQ ID NO:4, SEQ ID NO:5, SEQ ID NO:8, SEQ ID NO:9, SEQ ID NO:10, or a fragment thereof.

The predicted ECD domain for human VSTM4 is provided as SEQ ID NO:2 and SEQ ID NO:3, with and without the predicted signal sequence, respectively. The predicted ECD domain for mouse VSTM4 is provided as SEQ ID NO:7 and SEQ ID NO:8, with and without the predicted signal sequence, respectively. The predicted ECD domain for human B7-H4 is provided as SEQ ID NO:12 and SEQ ID NO:13, with and without the predicted signal sequence, respectively. The predicted ECD domain for mouse B7-H4 is provided as SEQ ID NO:17 and SEQ ID NO:18, with and without the predicted signal sequence, respectively. Those of skill in the art may differ in their understanding of the exact amino acids corresponding to the various extracellular domains or Ig domains. Thus, the N-terminus and/or C-terminus of an extracellular domain or Ig domain described herein may extend or be shortened by 1, 2, 3, 4, 5, 6, 7, 8, 9, 10 or more amino acids.

In some embodiments, the agent is a soluble receptor comprising a sequence selected from the group consisting of: SEQ ID NO:2, SEQ ID NO:3, SEQ ID NO:4, SEQ ID NO:5, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:17, SEQ ID NO:18, SEQ ID NO:19, and SEQ ID NO:20. In some embodiments, the agent is a soluble receptor comprising a fragment of a sequence selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20.

In certain embodiments, the agent is a soluble receptor comprising a variant of any one of the aforementioned VSTM4 ECD sequences or B7-H4 ECD sequences wherein the sequences comprise one or more (e.g., one, two, three, four, five, six, seven, eight, nine, ten, etc.) conservative substitutions and the agent retains binding capabilities.

In some embodiments, soluble receptors comprising a fragment of the extracellular domain of VSTM4 or B7-H4 can demonstrate altered biological activity (e.g., increased protein half-life) compared to soluble receptors comprising the entire VSTM4 ECD or B7-H4 ECD. In some embodiments, protein half-life can be further increased by covalent modification with polyethylene glycol (PEG) or polyethylene oxide (PEO).

In some embodiments, the binding agent, such as a soluble receptor, is a fusion protein. As used herein, a “fusion protein” is a hybrid protein expressed by a nucleic acid molecule comprising nucleotide sequences of at least two genes. In certain embodiments, a fusion protein which comprises the extracellular domain of a human VSTM4 protein, or a fragment thereof, further comprises a non-VSTM4 polypeptide (e.g., a heterologous polypeptide). In certain embodiments, a fusion protein which comprises the extracellular domain of B7-H4, or a fragment thereof, further comprises a non-B7-H4 polypeptide (e.g., a heterologous polypeptide). In some embodiments, fusion protein may include an extracellular domain, or fragment thereof, linked to heterologous functional and structural polypeptides including, but not limited to, a Fc region, a protein tag (e.g., myc, FLAG, GST, GFP), other endogenous proteins or protein fragments, or any other useful protein sequence including any linker region between the extracellular domain and the second polypeptide. In certain embodiments, the heterologous polypeptide is a Fc region. In certain embodiments, the heterologous polypeptide is a human Fc region. In certain embodiments, the heterologous polypeptide is a mouse Fc region. The Fc region can be obtained from any of the classes of immunoglobulin, IgG, IgA, IgM, IgD and IgE. In some embodiments, the Fc region is an IgG1 Fc region. In some embodiments, the Fc region is an IgG2 Fc region. In some embodiments, the Fc region is a wild-type Fc region. In some embodiments, the Fc region is a natural variant of a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, the Fc region is truncated at the C-terminal end (e.g., lysine is absent). In some embodiments, an amino acid in the hinge domain is changed to hinder undesirable disulfide bond formation. In some embodiments, a cysteine is replaced with a different amino acid to hinder undesirable disulfide bond formation. In some embodiments, a cysteine is replaced with a serine to hinder undesirable disulfide bond formation. In certain embodiments, the heterologous polypeptide comprises SEQ ID NO:21, SEQ ID NO:22, SEQ ID NO:23, SEQ ID NO:24, SEQ ID NO:25, or SEQ ID NO:26. In certain embodiments, the heterologous polypeptide consists essentially of SEQ ID NO:21, SEQ ID NO:22, SEQ ID NO:23, SEQ ID NO:24, SEQ ID NO:25, or SEQ ID NO:26.

In certain embodiments, an agent is a fusion protein comprising the extracellular domain of VSTM4, or a fragment thereof, and a Fc region. In certain embodiments, an agent is a fusion protein comprising the extracellular domain of B7-H4, or a fragment thereof, and a Fc region. In some embodiments, the C-terminus of the extracellular domain of VSTM4 or B7-H4 is linked to the N-terminus of the immunoglobulin Fc region. In some embodiments, the extracellular domain of VSTM4 or B7-H4 is directly linked to the Fc region (i.e. without an intervening peptide linker). In some embodiments, the extracellular domain of VSTM4 or B7-H4 is linked to the Fc region via a peptide linker.

As used herein, the term “linker” refers to a linker inserted between a first polypeptide (e.g., a VSTM4 ECD or fragment thereof) and a second polypeptide (e.g., a Fc region). In some embodiments, the linker is a peptide linker. Linkers should not adversely affect the expression, secretion, or bioactivity of the fusion protein. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. Linkers may include, but are not limited to, SerGly, GGSG, GSGS, GGGS, S(GGS)n where n is 1-7, GRA, poly(Gly), poly(Ala), ESGGGGVT (SEQ ID NO:32), LESGGGGVT (SEQ ID NO:33), GRAQVT (SEQ ID NO:34), WRAQVT (SEQ ID NO:35), and ARGRAQVT (SEQ ID NO:36). In some embodiments, the linker may comprise a cleavage site. In some embodiments, the linker may comprise an enzyme cleavage site, so that the second polypeptide may be separated from the first polypeptide. As used herein, a linker is an intervening peptide sequence that does not include amino acid residues from either the C-terminus of the first polypeptide (e.g., a VSTM4 ECD) or the N-terminus of the second polypeptide (e.g., the Fc region).

In some embodiments, the agent comprises a first polypeptide comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, or a fragment thereof, wherein the first polypeptide is directly linked to the second polypeptide.

In some embodiments, the agent comprises a first polypeptide comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, or a fragment thereof, wherein the first polypeptide is connected to the second polypeptide by a linker.

In some embodiments, the agent comprises a first polypeptide comprising SEQ ID NO:3, SEQ ID NO:4, SEQ ID NO:5, SEQ ID NO:8, SEQ ID NO:9, SEQ ID NO:10, SEQ ID NO:13, SEQ ID NO:14, SEQ ID NO:15, SEQ ID NO:18, SEQ ID NO:19, SEQ ID NO:20, or a fragment thereof, wherein the first polypeptide is directly linked to the second polypeptide.

In some embodiments, the agent comprises a first polypeptide comprising SEQ ID NO:3, SEQ ID NO:4, SEQ ID NO:5, SEQ ID NO:8, SEQ ID NO:9, SEQ ID NO:10, SEQ ID NO:13, SEQ ID NO:14, SEQ ID NO:15, SEQ ID NO:18, SEQ ID NO:19, SEQ ID NO:20, or a fragment thereof, wherein the first polypeptide is connected to the second polypeptide by a linker.

In some embodiments, the agent comprises a first polypeptide comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, or a fragment thereof; and a second polypeptide comprising SEQ ID NO:21, SEQ ID NO:22, SEQ ID NO:23, SEQ ID NO:24, SEQ ID NO:25, or SEQ ID NO:26, wherein the first polypeptide is directly linked to the second polypeptide. In some embodiments, the agent comprises a first polypeptide comprising SEQ ID NO:3, SEQ ID NO:4, SEQ ID NO:5, or a fragment thereof, and a second polypeptide comprising SEQ ID NO:21, SEQ ID NO:22, SEQ ID NO:23, or SEQ ID NO:24. In some embodiments, the agent comprises a first polypeptide comprising SEQ ID NO:13, SEQ ID NO:14, SEQ ID NO:15, or a fragment thereof, and a second polypeptide comprising SEQ ID NO:21, SEQ ID NO:22, SEQ ID NO:23, or SEQ ID NO:24. In some embodiments, the agent comprises a first polypeptide comprising SEQ ID NO:8, SEQ ID NO:9, SEQ ID NO:10, or a fragment thereof, and a second polypeptide comprising SEQ ID NO:25 or SEQ ID NO:26. In some embodiments, the agent comprises a first polypeptide comprising SEQ ID NO:18, SEQ ID NO:19, SEQ ID NO:20, or a fragment thereof, and a second polypeptide comprising SEQ ID NO:25 or SEQ ID NO:26.

In some embodiments, the agent comprises a first polypeptide comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, or a fragment thereof; and a second polypeptide comprising SEQ ID NO:21, SEQ ID NO:22, SEQ ID NO:23, SEQ ID NO:24, SEQ ID NO:25, or SEQ ID NO:26, wherein the first polypeptide is connected to the second polypeptide by a linker. In some embodiments, the agent comprises a first polypeptide comprising SEQ ID NO:3, SEQ ID NO:4, SEQ ID NO:5, or a fragment thereof, and a second polypeptide comprising SEQ ID NO:21, SEQ ID NO:22, SEQ ID NO:23, or SEQ ID NO:24. In some embodiments, the agent comprises a first polypeptide comprising SEQ ID NO:13, SEQ ID NO:14, SEQ ID NO:15, or a fragment thereof, and a second polypeptide comprising SEQ ID NO:21, SEQ ID NO:22, SEQ ID NO:23, or SEQ ID NO:24. In some embodiments, the agent comprises a first polypeptide comprising SEQ ID NO:8, SEQ ID NO:9, SEQ ID NO:10, or a fragment thereof, and a second polypeptide comprising SEQ ID NO:25 or SEQ ID NO:26. In some embodiments, the agent comprises a first polypeptide comprising SEQ ID NO:18, SEQ ID NO:19, SEQ ID NO:20, or a fragment thereof, and a second polypeptide comprising SEQ ID NO:25 or SEQ ID NO:26.

In some embodiments, the agent comprises a first polypeptide that is at least 80% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20; and a second polypeptide comprising SEQ ID NO:21, SEQ ID NO:22, SEQ ID NO:23, SEQ ID NO:24, SEQ ID NO:25, or SEQ ID NO:26, wherein the first polypeptide is directly linked to the second polypeptide. In some embodiments, the first polypeptide is at least 85%, at least 90%, at least 95%, 96%, 97%, 98% or 99% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20.

In some embodiments, the agent comprises a first polypeptide that is at least 80% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20; and a second polypeptide comprising SEQ ID NO:21, SEQ ID NO:22, SEQ ID NO:23, SEQ ID NO:24, SEQ ID NO:25, or SEQ ID NO:26, wherein the first polypeptide is connected to the second polypeptide by a linker. In some embodiments, the first polypeptide is at least 85%, at least 90%, at least 95%, 96%, 97%, 98% or 99% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20.

VSTM4 and B7-H4 are predicted to contain a signal sequence that directs the transport of the protein. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus. In some embodiments, the signal sequence of the polypeptide is not a native signal sequence.

In certain embodiments, an agent comprises a Fc region of an immunoglobulin. Those skilled in the art will appreciate that some of the binding agents of this invention will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region may include additions, deletions, or substitutions of one or more amino acids in one or more domains. The modified fusion proteins disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 aa residues) that provides some of the molecular flexibility typically imparted by the absent constant region domain.

In some embodiments, the modified fusion proteins are engineered to link the CH3 domain directly to the hinge region or to the first polypeptide. In other embodiments, a peptide spacer is inserted between the hinge region or the first polypeptide and the modified CH2 and/or CH3 domains. For example, constructs may be expressed wherein the CH2 domain has been deleted and the remaining CH3 domain (modified or unmodified) is joined to the hinge region or first polypeptide with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement C1q binding). Such partial deletions of the constant regions may improve selected characteristics of the binding agent (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors).

In some embodiments, the modified fusion proteins provide for altered effector functions that, in turn, affect the biological profile of the administered agent. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moiety attachment sites.

In certain embodiments, a modified fusion protein does not have one or more effector functions normally associated with an Fc region. In some embodiments, the agent has no ADCC activity, and/or no CDC activity. In certain embodiments, the agent does not bind to the Fc receptor and/or complement factors. In certain embodiments, the agent has no effector function.

The agents (e.g., antibodies or soluble receptors) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are well-known in the art (see, e.g., Ausubel et al., Editors, 1994-present, Current Protocols in Molecular Biology, John Wiley & Sons, Inc., New York, N.Y.).

For example, the specific binding of an agent (e.g., an antibody or a soluble receptor) to VSTM4 or B7-H4 may be determined using ELISA. An ELISA assay comprises preparing antigen, coating wells of a 96 well microtiter plate with antigen, adding the agent conjugated to a detectable compound such as an enzymatic substrate (e.g. horseradish peroxidase or alkaline phosphatase) to the well, incubating for a period of time and detecting the presence of the antibody bound to the antigen. In some embodiments, the agent is not conjugated to a detectable compound, but instead a second conjugated antibody that recognizes the agent is added to the well. In some embodiments, instead of coating the well with the antigen, the agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art.

In another example, the specific binding of an agent (e.g., an antibody or a soluble receptor) to VSTM4 or B7-H4 may be determined using FACS. A FACS screening assay may comprise generating a cDNA construct that expresses an antigen as a fusion protein (e.g., VSTM4-CD4TM), transfecting the construct into cells, expressing the antigen on the surface of the cells, mixing the agent with the transfected cells, and incubating for a period of time. The cells bound by the agent may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an agent (e.g., an antibody or a soluble receptor) to an antigen/target (e.g., VSTM4 or B7-H4) and the off-rate of a binding agent-antigen/target interaction can be determined by competitive binding assays. One example of a competitive binding assay is a radioimmunoassay comprising the incubation of labeled antigen/target (e.g., ³H or ¹²⁵I), or fragment or variant thereof, with the binding agent of interest in the presence of increasing amounts of unlabeled antigen/target followed by the detection of the binding agent bound to the labeled antigen/target. The affinity of the binding agent for an antigen/target (e.g., VSTM4 or B7-H4) and the binding off-rates can be determined from the data by Scatchard plot analysis. In some embodiments, Biacore kinetic analysis is used to determine the binding on and off rates of binding agents that bind an antigen/target (e.g., VSTM4 or B7-H4). Biacore kinetic analysis comprises analyzing the binding and dissociation of binding agents from chips with immobilized antigen/target (e.g., VSTM4 or B7-H4) on the chip surface.

This invention also encompasses heterodimeric molecules. Generally the heterodimeric molecule comprises two polypeptides. In some embodiments, the heterodimeric molecule is capable of binding at least two targets. The targets may be, for example, two different receptors on a single cell or two different receptors on two separate cells. Thus, in some embodiments, one polypeptide of the heterodimeric molecule comprises an antibody described herein (e.g., binds VSTM4) and one polypeptide of the heterodimeric molecule is a polypeptide that may be a fusion protein or a soluble receptor. In some embodiments, the heterodimeric molecule is capable of binding one target and also comprises a “non-binding” function. Thus in some embodiments, one polypeptide of the heterodimeric molecule comprises a polypeptide described herein (e.g., VSTM4-binding agent) and one polypeptide of the heterodimeric molecule is an immune response stimulating agent. As used herein, the phrase “immune response stimulating agent” is used in the broadest sense and refers to a substance that directly or indirectly stimulates the immune system by inducing activation or increasing activity of any of the immune system's components. For example, immune response stimulating agents include cytokines, as well as various antigens including tumor antigens, and antigens derived from pathogens. In some embodiments, the immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), an antibody that blocks immunosuppressive functions (e.g., an anti-CTLA4 antibody, anti-CD28 antibody, anti-CD3 antibody), a toll-like receptor (e.g., TLR4, TLR7, TLR9), or a member of the B7 family (e.g., CD80, CD86).

In some embodiments, the heterodimeric molecule can bind a first target, (e.g., VSTM4) as well as a second target, such as an effector molecule on a different immune cell (e.g., CD2, CD3, CD28, or CD80) or a Fc receptor (e.g., CD64, CD32, or CD16) so as to elicit a stronger cellular immune response.

In some embodiments, a heterodimeric molecule has enhanced potency as compared to an individual agent. It is known to those of skill in the art that any agent (e.g., an antibody or a soluble receptor) may have unique pharmacokinetics (PK) (e.g., circulating half-life). In some embodiments, a heterodimeric molecule has the ability to synchronize the PK of two active agents and/or polypeptides wherein the two individual agents and/or polypeptides have different PK profiles. In some embodiments, a heterodimeric molecule has the ability to concentrate the actions of two agents and/or polypeptides in a common area (e.g., a tumor and/or tumor environment). In some embodiments, a heterodimeric molecule has the ability to concentrate the actions of two agents and/or polypeptides to a common target (e.g., a tumor or a tumor cell). In some embodiments, a heterodimeric molecule has the ability to target the actions of two agents and/or polypeptides to more than one biological pathway or more than one aspect of the immune response. In some embodiments, the heterodimeric molecule has decreased toxicity and/or side effects than either of the agents and/or polypeptides alone. In some embodiments, the heterodimeric molecule has decreased toxicity and/or side effects as compared to a mixture of the two individual agents and/or polypeptides. In some embodiments, the heterodimeric molecule has an increased therapeutic index. In some embodiments, the heterodimeric molecule has an increased therapeutic index as compared to a mixture of the two individual agents and/or polypeptides or the agents and/or polypeptides as single agents.

In some embodiments, the binding agent is a heterodimeric molecule which comprises a first CH3 domain and a second CH3 domain, each of which is modified to promote formation of heteromultimers or heterodimers. In some embodiments, the first and second CH3 domains are modified using a knobs-into-holes technique. In some embodiments, the first and second CH3 domains comprise changes in amino acids that result in altered electrostatic interactions. In some embodiments, the first and second CH3 domains comprise changes in amino acids that result in altered hydrophobic/hydrophilic interactions (see, for example, U.S. Patent App. Publication No. 2011/0123532).

In some embodiments, the binding agent (e.g., antibody or soluble receptor) is a heterodimeric molecule which comprises heavy chain constant regions selected from the group consisting of: (a) a first human IgG1 constant region, wherein the amino acids at positions corresponding to positions 253 and 292 of SEQ ID NO:27 are replaced with glutamate or aspartate, and a second human IgG1 constant region, wherein the amino acids at positions corresponding to 240 and 282 of SEQ ID NO:27 are replaced with lysine; (b) a first human IgG2 constant region, wherein the amino acids at positions corresponding to positions 249 and 288 of SEQ ID NO:28 are replaced with glutamate or aspartate, and a second human IgG2 constant region wherein the amino acids at positions corresponding to positions 236 and 278 of SEQ ID NO:28 are replaced with lysine; (c) a first human IgG3 constant region, wherein the amino acids at positions corresponding to positions 300 and 339 of SEQ ID NO:29 are replaced with glutamate or aspartate, and a second human IgG3 constant region wherein the amino acids at positions corresponding to positions 287 and 329 of SEQ ID NO:29 are replaced with lysine; and (d) a first human IgG4 constant region, wherein the amino acids at positions corresponding to positions 250 and 289 of SEQ ID NO:30 are replaced with glutamate or aspartate, and a second IgG4 constant region wherein the amino acids at positions corresponding to positions 237 and 279 of SEQ ID NO:30 are replaced with lysine.

In some embodiments, the heterodimeric protein comprises two polypeptides, wherein each polypeptide comprises a human IgG1 CH3 domain, and wherein the amino acids at positions corresponding to positions 253 and 292 of SEQ ID NO:27 of one IgG1 CH3 domain are replaced with glutamate or aspartate, and wherein the amino acids at positions corresponding to positions 240 and 282 of SEQ ID NO:27 of the other IgG1 CH3 domain are replaced with lysine.

In some embodiments, the heterodimeric protein comprises two polypeptides, wherein each polypeptide comprises a human IgG2 CH3 domain, and wherein the amino acids at positions corresponding to positions 249 and 288 of SEQ ID NO:28 of one IgG2 CH3 domain are replaced with glutamate or aspartate, and wherein the amino acids at positions corresponding to positions 236 and 278 of SEQ ID NO:28 of the other IgG2 CH3 domain are replaced with lysine.

In some embodiments, the binding agent (e.g., a soluble receptor) is a heterodimeric molecule which comprises a first human IgG1 constant region with amino acid substitutions at positions corresponding to positions 253 and 292 of SEQ ID NO:27, wherein the amino acids are replaced with glutamate or aspartate, and a second human IgG1 constant region with amino acid substitutions at positions corresponding to positions 240 and 282 of SEQ ID NO:27, wherein the amino acids are replaced with lysine. In some embodiments, the binding agent (e.g., a soluble receptor) is a fusion protein which comprises a first human IgG2 constant region with amino acid substitutions at positions corresponding to positions 249 and 288 of SEQ ID NO:28, wherein the amino acids are replaced with glutamate or aspartate, and a second human IgG2 constant region with amino acid substitutions at positions corresponding to positions 236 and 278 of SEQ ID NO:28, wherein the amino acids are replaced with lysine. In some embodiments, the binding agent (e.g., a soluble receptor) is a fusion protein which comprises a first human IgG3 constant region with amino acid substitutions at positions corresponding to positions 300 and 339 of SEQ ID NO:29, wherein the amino acids are replaced with glutamate or aspartate, and a second human IgG3 constant region with amino acid substitutions at positions corresponding to positions 287 and 329 of SEQ ID NO:29, wherein the amino acids are replaced with lysine. In some embodiments, the binding agent (e.g., a soluble receptor) is a fusion protein which comprises a first human IgG4 constant region with amino acid substitutions at positions corresponding to positions 250 and 289 of SEQ ID NO:30, wherein the amino acids are replaced with glutamate or aspartate, and a second human IgG4 constant region with amino acid substitutions at positions corresponding to positions 237 and 279 of SEQ ID NO:30, wherein the amino acids are replaced with lysine.

In some embodiments, the binding agent (e.g., a soluble receptor) is a fusion protein which comprises a first human IgG1 constant region with amino acid substitutions at positions corresponding to positions 253 and 292 of SEQ ID NO:27, wherein the amino acids are replaced with glutamate, and a second human IgG1 constant region with amino acid substitutions at positions corresponding to positions 240 and 282 of SEQ ID NO:27, wherein the amino acids are replaced with lysine. In some embodiments, the binding agent (e.g., a soluble receptor) is a fusion protein which comprises a first human IgG1 constant region with amino acid substitutions at positions corresponding to positions 253 and 292 of SEQ ID NO:27, wherein the amino acids are replaced with aspartate, and a second human IgG1 constant region with amino acid substitutions at positions corresponding to positions 240 and 282 of SEQ ID NO:27, wherein the amino acids are replaced with lysine.

In some embodiments, the binding agent (e.g., a soluble receptor) is a fusion protein which comprises a first human IgG2 constant region with amino acid substitutions at positions corresponding to positions 249 and 288 of SEQ ID NO:28, wherein the amino acids are replaced with glutamate, and a second human IgG2 constant region with amino acid substitutions at positions corresponding to positions 236 and 278 of SEQ ID NO:28, wherein the amino acids are replaced with lysine. In some embodiments, the binding agent (e.g., a soluble receptor) is a fusion protein which comprises a first human IgG2 constant region with amino acid substitutions at positions corresponding to positions 249 and 288 of SEQ ID NO:28, wherein the amino acids are replaced with aspartate, and a second human IgG2 constant region with amino acid substitutions at positions corresponding to positions 236 and 278 of SEQ ID NO:28, wherein the amino acids are replaced with lysine.

In some embodiments, the binding agents described herein are monovalent. In some embodiments, the binding agent is a heterodimeric protein that is monovalent. In some embodiments, the binding agent is a soluble receptor that is monovalent. In some embodiments, the binding agents described herein are bivalent. In some embodiments, the binding agents described herein are monospecific. In some embodiments, the binding agents described herein are bispecific. In some embodiments, the binding agents described herein are multispecific.

In some embodiments, the binding agents are substantially homologous to the soluble receptors, fusion proteins, and/or polypeptides described herein. In some embodiments, the binding agents contain conservative substitution mutations.

In certain embodiments, the agents described herein bind VSTM4 and modulate an immune response. In certain embodiments, the agents described herein bind B7-H4 and modulate an immune response. In some embodiments, an agent (e.g., an antibody or a soluble receptor) activates and/or increases an immune response. In some embodiments, an agent induces, activates, increases, promotes, enhances, and/or prolongs cell-mediated immunity. In some embodiments, an agent induces, activates, increases, promotes, enhances, and/or prolongs innate cell-mediated immunity. In some embodiments, an agent induces, activates, increases, promotes, enhances, and/or prolongs adaptive cell-mediated immunity. In some embodiments, an agent induces, activates, increases, promotes, enhances, and/or prolongs T-cell activity. In some embodiments, an agent induces, activates, increases, promotes, enhances, and/or prolongs cytolytic T-cell (CTL) activity. In some embodiments, an agent induces, activates, increases, promotes, enhances, and/or prolongs NK cell activity. In some embodiments, an agent induces, activates, increases, promotes, enhances, and/or prolongs lymphokine-activated killer cell (LAK) activity. In some embodiments, an agent induces, activates, increases, promotes, enhances, and/or prolongs tumor cell killing. In some embodiments, an agent induces, activates, increases, promotes, enhances, and/or prolongs the inhibition of tumor growth.

In some embodiments, the agents described herein inhibit VSTM4 activity. In some embodiments, the agents described herein inhibit the inhibitory or suppressive activity of VSTM4. In some embodiments, the agents described herein block VSTM4 activity.

In some embodiments, the agents described herein bind VSTM4 or B7-H4 and inhibit VSTM4 signaling. In some embodiments, an agent binds VSTM4 and inhibits VSTM4 signaling. In some embodiments, an agent binds B7-H4 and inhibits VSTM4 signaling. In some embodiments, an agent binds VSTM4 and blocks VSTM4 signaling. In some embodiments, an agent binds B7-H4 and blocks VSTM4 signaling.

In some embodiments, an agent described herein binds VSTM4, wherein the agent disrupts binding of VSTM4 to B7-H4; and/or disrupts B7-H4 activation of VSMT4 signaling. In some embodiments, the agent disrupts binding of VSTM4 to B7-H4. In some embodiments, the agent disrupts B7-H4 activation of VSTM4 signaling. In some embodiments, an agent binds B7-H4, wherein the agent disrupts binding of B7-H4 to VSTM4; and/or disrupts B7-H4 activation of VSTM4 signaling. In some embodiments, the agent disrupts binding of B7-H4 to VSTM4. In some embodiments, the agent disrupts B7-H4 activation of VSTM4 signaling. In some embodiments, the disruption induces, activates, increases, promotes, enhances, and/or prolongs an immune response.

In certain embodiments, an agent described herein is an antagonist (either directly or indirectly) of VSTM4. In certain embodiments, an agent described herein is an antagonist (either directly or indirectly) of VSTM4 which comprises an immunoreceptor tyrosine-based inhibitory motif (ITIM). An antagonist of VSTM4 may act as a “brake” on the inhibitory activity. Therefore, in some embodiments, the agent is an antagonist of VSTM4 and induces and/or enhances an immune response. In some embodiments, the binding agent is an antagonist of VSTM4 and induces and/or enhances activity of NK cells and/or T-cells (e.g., cytolytic activity or cytokine production). In certain embodiments, the binding agent induces and/or enhances the activity by at least about 10%, at least about 20%, at least about 30%, at least about 50%, at least about 75%, at least about 90%, or about 100%. In some embodiments, the binding agent decreases or inhibits the activity of Treg cells. In certain embodiments, the binding agent decreases or inhibits the activity by at least about 10%, at least about 20%, at least about 30%, at least about 50%, at least about 75%, at least about 90%, or about 100%. In some embodiments, the antagonist of VSTM4 is an antibody described herein that specifically binds to the extracellular domain, or a fragment thereof, of VSTM4.

In certain embodiments, an agent described herein is a soluble receptor comprising the extracellular domain, or a fragment thereof, of VSTM4. A soluble receptor comprising the extracellular domain, or a fragment thereof, of VSTM4 may act as a “receptor decoy” of B7-H4. In certain embodiments, a soluble receptor comprising the extracellular domain, or a fragment thereof, of VSTM4 binds and/or interacts with B7-H4, which inhibits or blocks the interaction of B7-H4 with VSTM4 on a cell. Therefore, in some embodiments, the agent is an antagonist of B7-H4 and induces and/or enhances an immune response. In some embodiments, the agent is an antagonist of B7-H4 and induces and/or enhances activity of NK cells and/or T-cells (e.g., cytolytic activity or cytokine production). In certain embodiments, the binding agent induces and/or enhances the activity by at least about 10%, at least about 20%, at least about 30%, at least about 50%, at least about 75%, at least about 90%, or about 100%. In some embodiments, the binding agent decreases or inhibits the activity of Treg cells. In certain embodiments, the binding agent decreases or inhibits the activity by at least about 10%, at least about 20%, at least about 30%, at least about 50%, at least about 75%, at least about 90%, or about 100%.

In certain embodiments, an agent described herein increases activation of a NK cell. In certain embodiments, an agent increases activation of a T-cell. In certain embodiments, the activation of a NK cell and/or a T-cell by an agent results in an increase in the level of activation of a NK cell and/or a T-cell of at least about 10%, at least about 25%, at least about 50%, at least about 75%, at least about 90%, or at least about 95%.

In vivo and in vitro assays for determining whether a binding agent (or candidate binding agent) modulates an immune response are known in the art or are being developed. In some embodiments, a functional assay that detects T-cell activation may be used. In some embodiments, a functional assay that detects T-cell proliferation may be used. In some embodiments, a functional assay that detects lymphokine production may be used.

In certain embodiments, the binding agents are capable of inhibiting tumor growth. In certain embodiments, the binding agents are capable of inhibiting tumor growth in vivo (e.g., in a xenograft mouse model, and/or in a human having cancer).

In certain embodiments, the binding agents are capable of reducing the tumorigenicity of a tumor. In certain embodiments, the binding agent is capable of reducing the tumorigenicity of a tumor in an animal model, such as a mouse xenograft model. In certain embodiments, the binding agent is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236; U.S. Patent Publication No. 2008/0064049; and U.S. Patent Publication No. 2008/0178305.

In certain embodiments, the binding agents have one or more of the following effects: inhibit proliferation of tumor cells, inhibit tumor growth, reduce the tumorigenicity of a tumor, reduce the tumorigenicity of a tumor by reducing the frequency of cancer stem cells in the tumor, trigger cell death of tumor cells, increase cell contact-dependent growth inhibition, increase tumor cell apoptosis, reduce epithelial mesenchymal transition (EMT), or decrease survival of tumor cells. In some embodiments, the binding agents have one or more of the following effects: inhibit viral infection, inhibit chronic viral infection, reduce viral load, trigger cell death of virus-infected cells, or reduce the number or percentage of virus-infected cells.

In certain embodiments, a binding agent described herein has a circulating half-life in mice, cynomolgus monkeys, or humans of at least about 5 hours, at least about 10 hours, at least about 24 hours, at least about 3 days, at least about 1 week, or at least about 2 weeks. In certain embodiments, the binding agent is an IgG (e.g., IgG1 or IgG2) fusion protein that has a circulating half-life in mice, cynomolgus monkeys, or humans of at least about 5 hours, at least about 10 hours, at least about 24 hours, at least about 3 days, at least about 1 week, or at least about 2 weeks. Methods of increasing (or decreasing) the half-life of agents such as polypeptides and soluble receptors are known in the art. For example, known methods of increasing the circulating half-life of IgG fusion proteins include the introduction of mutations in the Fc region which increase the pH-dependent binding of the antibody to the neonatal Fc receptor (FcRn) at pH 6.0. Known methods of increasing the circulating half-life of soluble receptors lacking a Fc region include such techniques as PEGylation.

In some embodiments of the present invention, the binding agents are polypeptides. The polypeptides can be recombinant polypeptides, natural polypeptides, or synthetic polypeptides that bind VSTM4 or B7-H4. It will be recognized in the art that some amino acid sequences of the invention can be varied without significant effect of the structure or function of the protein. Thus, the invention further includes variations of the polypeptides which show substantial binding activity to VSTM4 or B7-H4. In some embodiments, amino acid sequence variations of the polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 22^{st} Edition, 2012, Pharmaceutical Press, London.

The polypeptides described herein, including antibodies, soluble receptors, and fusion proteins, can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof by methods know in the art.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding the binding agents (e.g., antibodies or soluble receptors) described herein. For example, recombinant expression vectors can be replicable DNA constructs which have synthetic or cDNA-derived DNA fragments encoding a polypeptide chain of a binding agent operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a polypeptide (or a protein to use as an antigen or as a target) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are well-known. Additional information regarding methods of protein production, including antibody production, can be found, e.g., in U.S. Patent Publication No. 2008/0187954; U.S. Pat. Nos. 6,413,746 and 6,660,501; and International Patent Publication No. WO 2004/009823.

Various mammalian cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), and HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present invention provides cells comprising the binding agents described herein. In some embodiments, the cells produce the binding agents described herein. In certain embodiments, the cells produce a fusion protein. In some embodiments, the cells produce a soluble receptor. In some embodiments, the cells produce an antibody. In some embodiments, the cells produce a bispecific antibody. In some embodiments, the cells produce a heterodimeric protein.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

In certain embodiments, a binding agent described herein is a polypeptide that does not comprise an immunoglobulin Fc region. In certain embodiments, the polypeptide comprises a protein scaffold of a type selected from the group consisting of protein A, protein G, a lipocalin, a fibronectin domain, an ankyrin consensus repeat domain, and thioredoxin. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify a binding polypeptide. In certain embodiments, mammalian cell display technology may be used to produce and/or identify a binding polypeptide.

It can further be desirable to modify a polypeptide in order to increase (or decrease) its serum half-life. This can be achieved, for example, by incorporation of a salvage receptor binding epitope into the polypeptide by mutation of the appropriate region in the polypeptide or by incorporating the epitope into a peptide tag that is then fused to the polypeptide at either end or in the middle (e.g., by DNA or peptide synthesis).

Heteroconjugate molecules are also within the scope of the present invention. Heteroconjugate molecules are composed of two covalently joined polypeptides. Such molecules have, for example, been proposed to target immune cells to unwanted cells, such as tumor cells. It is also contemplated that the heteroconjugate molecules can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

In certain embodiments, a binding agent described herein can be used in any one of a number of conjugated (i.e. an immunoconjugate or radioconjugate) or non-conjugated forms. In certain embodiments, the binding agents can be used in a non-conjugated form to harness the subject's natural defense mechanisms including CDC and ADCC to eliminate malignant or cancer cells.

In certain embodiments, a binding agent described herein is a small molecule. The term “small molecule” generally refers to a low molecular weight organic compound which is by definition not a peptide/protein.

In some embodiments, a binding agent described herein is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated binding agent. A variety of radionuclides are available for the production of radioconjugated binding agents including, but not limited to, ⁹⁰Y, ¹²⁵I, ¹³¹I, ¹²³I, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re, and ²¹²Bi. Conjugates of a binding agent and one or more small molecule toxins, such as a calicheamicin, maytansinoids, a trichothene, and CC1065, and the derivatives of these toxins that have toxin activity, can also be used. Conjugates of a binding agent and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. Polynucleotides

In certain embodiments, the invention encompasses polynucleotides comprising polynucleotides that encode a binding agent (e.g., an antibody or a soluble receptor) described herein. The term “polynucleotides that encode a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, a polynucleotide comprises a polynucleotide encoding an antibody that specifically binds the extracellular domain of VSTM4 or a fragment thereof. In some embodiments, a polynucleotide comprises a polynucleotide encoding an antibody that specifically binds SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a fragment thereof.

In some embodiments, a polynucleotide comprises a polynucleotide encoding an antibody that specifically binds the extracellular domain of B7-H4 or a fragment thereof. In some embodiments, a polynucleotide comprises a polynucleotide encoding an antibody that specifically binds SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, or a fragment thereof.

In some embodiments, a polynucleotide comprises a polynucleotide encoding a soluble receptor that binds B7-H4. In some embodiments, a polynucleotide comprises a polynucleotide encoding a soluble receptor that binds VSTM4.

In certain embodiments, a polynucleotide comprises a polynucleotide encoding a polypeptide comprising an amino acid sequence selected from the group consisting of: SEQ ID NOs:1-20 or a fragment thereof.

In certain embodiments, a polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide encoding an amino acid sequence selected from the group consisting of SEQ ID NOs:1-20, or a fragment thereof. Also provided is a polynucleotide that comprises a polynucleotide that hybridizes to a polynucleotide encoding an amino acid sequence selected from the group consisting of SEQ ID NOs:1-20, or a fragment thereof. In certain embodiments, the hybridization is under conditions of high stringency. Conditions of high stringency are known to those of skill in the art and may include but are not limited to, (1) employ low ionic strength and high temperature for washing, for example 15 mM sodium chloride/1.5 mM sodium citrate (1×SSC) with 0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes in 0.2×SSC containing 50% formamide at 55° C., followed by a high-stringency wash consisting of 0.1×SSC containing EDTA at 55° C.

In certain embodiments, a polynucleotide comprises the coding sequence for a mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments, a polynucleotide comprises the coding sequence for a mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag, a peptide of sequence DYKDDDDK (SEQ ID NO:31) which can be used in conjunction with other affinity tags.

The present invention further relates to variants of the hereinabove described polynucleotides encoding, for example, fragments, analogs, and/or derivatives.

In certain embodiments, the present invention provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a binding agent (e.g., an antibody, a soluble receptor, or a polypeptide) described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In some embodiments, at least one polynucleotide variant is produced (without changing the amino acid sequence of the encoded polypeptide) to increase production of a heterodimeric molecule. In some embodiments, at least one polynucleotide variant is produced (without changing the amino acid sequence of the encoded polypeptide) to increase production of a bispecific antibody.

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

### IV. Methods of Use

The binding agents of the invention are useful in a variety of applications including, but not limited to, therapeutic treatment methods, such as immunotherapy for cancer. In certain embodiments, the binding agents are useful for inducing, activating, promoting, increasing, enhancing, and/or prolonging an immune response. In certain embodiments, the binding agents are useful for inducing, activating, promoting, increasing, enhancing, and/or prolonging an immune response to a tumor or a tumor cell. In certain embodiments, the binding agents are useful for inhibiting tumor growth, reducing tumor volume, increasing tumor cell apoptosis, and/or reducing the tumorigenicity of a tumor. The binding agents of the invention are also useful for immunotherapy against pathogens, such as viruses. In certain embodiments, the binding agents are useful for inducing, activating, promoting, increasing, and/or enhancing an immune response to a virus, inhibiting viral infection, reducing viral infection, increasing virally-infected cell apoptosis, and/or increasing killing of virus-infected cells. The methods of use may be in vitro, ex vivo, or in vivo methods.

The present invention provides methods of modulating an immune response. In some embodiments, the invention provides a method of modulating the activity of immune cells comprising contacting the cells with an effective amount of an agent described herein. In some embodiments, the immune cells are T-cells, NK cells, monocytes, macrophages, and/or B-cells. In some embodiments, the invention provides a method of inducing an immune response in a subject using a binding agent described herein. In some embodiments, the invention provides a method of activating an immune response in a subject using a binding agent described herein. In some embodiments, the invention provides a method of promoting an immune response in a subject using a binding agent described herein. In some embodiments, the invention provides a method of increasing an immune response in a subject using a binding agent described herein. In some embodiments, the invention provides a method of enhancing an immune response in a subject using a binding agent described herein. In some embodiments, the invention provides a method of prolonging an immune response in a subject using a binding agent described herein. In some embodiments, a method comprises administering to a subject a therapeutically effective amount of an agent described herein.

In some embodiments, the inducing, activating, promoting, increasing, enhancing, and/or prolonging of an immune response comprises increasing cell-mediated immunity. In some embodiments, the activating, promoting, increasing, enhancing, and/or prolonging of an immune response comprises increasing T-cell activity. In some embodiments, the activating, promoting, increasing, enhancing, and/or prolonging of an immune response comprises inhibiting Treg activity. In some embodiments, the activating, promoting, increasing, enhancing, and/or prolonging of an immune response comprises increasing CTL activity. In some embodiments, the activating, promoting, increasing, enhancing, and/or prolonging of an immune response comprises increasing NK cell activity. In some embodiments, the activating, promoting, increasing, enhancing, and/or prolonging of an immune response comprises increasing T-cell activity and increasing NK cell activity. In some embodiments, the activating, promoting, increasing, enhancing, and/or prolonging of an immune response comprises increasing CTL activity and increasing NK cell activity. In some embodiments, the immune response is a result of antigenic stimulation. In some embodiments, the antigenic stimulation is a tumor or a tumor cell. In some embodiments, the antigenic stimulation is cancer. In some embodiments, the antigenic stimulation is a pathogen. In some embodiments, the antigenic stimulation is a virus. In some embodiments, the antigenic stimulation is a virally-infected cell.

In some embodiments, a method of inducing, activating, promoting, increasing, and/or enhancing an immune response in a subject comprises administering to the subject a therapeutically effective amount of an agent described herein. In some embodiments of the method, an agent disrupts the signaling of a B7-H4/VSTM4 pathway. In some embodiments of the method, the disruption of the signaling of a B7-H4/VSTM4 pathway inhibits the B7-H4-mediated suppression of immune responses. In some embodiments of the method, an agent that specifically binds the extracellular domain of VSTM4 disrupts signaling of a B7-H4/VSTM4 pathway. In some embodiments of the method, an antibody that specifically binds the extracellular domain of VSTM4 disrupts signaling of a B7-H4/VSTM4 pathway. In some embodiments of the method, an agent that specifically binds the extracellular domain of B7-H4 disrupts signaling of a B7-H4/VSTM4 pathway. In some embodiments of the method, an antibody that specifically binds the extracellular domain of B7-H4 disrupts signaling of a B7-H4/VSTM4 pathway. In some embodiments of the method, a soluble receptor that specifically binds the extracellular domain of B7-H4 disrupts signaling of a B7-H4/VSTM4 pathway.

In some embodiments, a method of inducing, activating, promoting, increasing, and/or enhancing an immune response in a subject comprises administering to the subject a therapeutically effective amount of an agent described herein, wherein the agent inhibits the interaction between VSTM4 and B7-H4. In some embodiments, a method of inducing, activating, promoting, increasing, and/or enhancing an immune response in a subject comprises administering to the subject a therapeutically effective amount of an agent described herein, wherein the agent is an antibody that specifically binds VSTM4. In some embodiments, a method of inducing, activating, promoting, increasing, and/or enhancing an immune response in a subject comprises administering to the subject a therapeutically effective amount of an agent described herein, wherein the agent is an antibody that specifically binds B7-H4. In some embodiments, a method of inducing, activating, promoting, increasing, and/or enhancing an immune response in a subject comprises administering to the subject a therapeutically effective amount of an agent described herein, wherein the agent is a soluble receptor that specifically binds B7-H4.

The present invention also provides methods for inhibiting growth of a tumor using the binding agents described herein. In certain embodiments, the method of inhibiting growth of a tumor comprises contacting a cell mixture with a binding agent described herein in vitro. For example, an immortalized cell line or a cancer cell line mixed with immune cells (e.g., T-cells or NK cells) is cultured in medium to which is added a binding agent. In some embodiments, tumor cells are isolated from a patient sample such as, for example, a tissue biopsy, pleural effusion, or blood sample, mixed with immune cells (e.g., T-cells and/or NK cells), and cultured in medium to which is added a binding agent. In some embodiments, the binding agent induces, increases, promotes, and/or enhances the activity of the immune cells. In some embodiments, the binding agent inhibits tumor cell growth. In some embodiments, the binding agent is a soluble receptor. In some embodiments, the binding agent is an antibody. In some embodiments, the agent binds VSTM4. In some embodiments, the agent binds B7-H4. In some embodiments, the agent is an antibody that specifically binds VSTM4. In some embodiments, the agent is an antibody that specifically binds B7-H4. In some embodiments, the agent is a soluble receptor that binds B7-H4.

In some embodiments, a method of inhibiting growth of a tumor comprises contacting a cell mixture with an agent that specifically binds the extracellular domain of VSTM4, wherein the agent disrupts binding of VSTM4 to B7-H4; and/or disrupts B7-H4 activation of VSTM4 signaling. In some embodiments, a method of inhibiting growth of a tumor comprises contacting a cell mixture with an agent that specifically binds the extracellular domain of B7-H4, wherein the agent disrupts binding of B7-H4 to VSTM4; and/or disrupts B7-H4 activation of VSTM4 signaling.

In some embodiments, the method of inhibiting growth of a tumor comprises contacting the tumor or tumor cells with a binding agent described herein in vivo. In certain embodiments, contacting a tumor or tumor cell with a binding agent is undertaken in an animal model. For example, a binding agent may be administered to mice which have syngeneic tumors. In some embodiments, the binding agent induces, increases, promotes, enhances, and/or prolongs the activity of immune cells in the mice. In some embodiments, the binding agent inhibits tumor growth. In some embodiments, the binding agent is administered at the same time or shortly after introduction of tumor cells into the animal to prevent tumor growth (“preventative model”). In some embodiments, the binding agent is administered as a therapeutic after tumors have grown to a specified size (“therapeutic model”). In some embodiments, the binding agent is a soluble receptor. In some embodiments, the binding agent is an antibody. In some embodiments, the binding agent is a polypeptide.

In certain embodiments, a method of inhibiting growth of a tumor in a subject comprises administering to the subject a therapeutically effective amount of a binding agent described herein. In certain embodiments, the subject is a human. In certain embodiments, the subject has a tumor or has had a tumor which was removed. In some embodiments, the binding agent is a soluble receptor. In some embodiments, the binding agent is an antibody. In some embodiments, the binding agent is a polypeptide. In some embodiments, the agent is an antibody that specifically binds VSTM4. In some embodiments, the agent is an antibody that specifically binds B7-H4. In some embodiments, the agent is a soluble receptor that binds B7-H4.

In some embodiments, a method of inhibiting growth of a tumor in a subject comprises administering to the subject a therapeutically effective amount of an agent that specifically binds the extracellular domain of VSTM4, wherein the agent disrupts binding of VSTM4 to B7-H4; and/or disrupts B7-H4 activation of VSTM4 signaling. In some embodiments, a method of inhibiting growth of a tumor in a subject comprises administering to the subject a therapeutically effective amount of an agent that specifically binds the extracellular domain of B7-H4, wherein the agent disrupts binding of B7-H4 to VSTM4; and/or disrupts B7-H4 activation of VSTM4 signaling. In some embodiments, the agent is an antibody that specifically binds VSTM4. In some embodiments, the agent is an antibody that specifically binds B7-H4. In some embodiments, the agent is a soluble receptor that binds B7-H4.

In addition, the invention provides a method of inhibiting growth of a tumor in a subject, comprising administering to the subject a therapeutically effective amount of a binding agent described herein, wherein the tumor comprises cancer stem cells. In certain embodiments, the frequency of cancer stem cells in the tumor is reduced by administration of the binding agent. In some embodiments, a method of reducing the frequency of cancer stem cells in a tumor in a subject, comprising administering to the subject a therapeutically effective amount of a binding agent is provided. In some embodiments, the binding agent is a soluble receptor. In some embodiments, the binding agent is an antibody. In some embodiments, the binding agent is a polypeptide. In some embodiments, the agent is an antibody that specifically binds VSTM4, or a fragment thereof. In some embodiments, the agent is an antibody that specifically binds B7-H4, or a fragment thereof. In some embodiments, the agent is a soluble receptor that binds B7-H4.

In addition, the invention provides a method of reducing the tumorigenicity of a tumor in a subject, comprising administering to the subject a therapeutically effective amount of a binding agent described herein. In certain embodiments, the tumor comprises cancer stem cells. In some embodiments, the tumorigenicity of a tumor is reduced by reducing the frequency of cancer stem cells in the tumor.

In some embodiments, the tumor is a solid tumor. In certain embodiments, the tumor is a tumor selected from the group consisting of: colorectal tumor, colon tumor, pancreatic tumor, lung tumor, ovarian tumor, liver tumor, breast tumor, kidney tumor, prostate tumor, neuroendocrine tumor, gastric tumor, gastrointestinal tumor, melanoma, cervical tumor, bladder tumor, glioblastoma, brain tumor, and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic tumor. In certain embodiments, the tumor is a melanoma tumor.

The present invention further provides methods for treating cancer in a subject comprising administering to the subject a therapeutically effective amount of an agent described herein. In some embodiments, the agent binds the extracellular domain, or a fragment thereof, of VSTM4. In some embodiments, the agent binds the extracellular domain, or a fragment thereof, of B7-H4. In some embodiments, the agent increases an immune response, and inhibits or reduces growth of the cancer. In some embodiments, the agent comprises an antibody that binds VSTM4. In some embodiments, the agent comprises an antibody that binds B7-H4. In some embodiments, the binding agent comprises a soluble receptor that binds B7-H4.

The present invention provides for methods of treating cancer comprising administering a therapeutically effective amount of a binding agent described herein to a subject (e.g., a subject in need of treatment). In certain embodiments, the subject is a human. In certain embodiments, the subject has a cancerous tumor. In certain embodiments, the subject has had a tumor removed.

In certain embodiments, the cancer is a cancer selected from the group consisting of colorectal cancer, colon cancer, pancreatic cancer, lung cancer, ovarian cancer, liver cancer, breast cancer, kidney cancer, prostate cancer, gastrointestinal cancer, gastric cancer, melanoma, cervical cancer, neuroendocrine cancer, bladder cancer, glioblastoma, brain cancer, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma.

In some embodiments, the cancer is a hematologic cancer. In some embodiments, the cancer is a T-cell hematologic cancer. In some embodiments, the cancer is a B-cell hematologic cancer. Hematologic cancers include, but are not limited to, acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

The invention also provides a method of inactivating, inhibiting, suppressing, or blocking VSTM4 signaling in a cell comprising contacting the cell with an effective amount of an agent described herein. In certain embodiments, the cell is a T-cell. In some embodiments, the cell is a cytolytic cell. In some embodiments, the cell is a CTL. In some embodiments, the cell is a NK cell. In certain embodiments, the method is an in vivo method wherein the step of contacting the cell with the binding agent comprises administering a therapeutically effective amount of the binding agent to the subject. In some embodiments, the method is an in vitro or ex vivo method. In some embodiments, the binding agent is a soluble receptor. In some embodiments, the binding agent is a polypeptide. In some embodiments, the binding agent is an antibody.

The invention also provides a method of stimulating a protective response in a subject comprising administering to the subject a therapeutically effective amount of an agent described herein in combination with an antigen of interest. In some embodiments, the antigen of interest is a tumor antigen. In some embodiments, the antigen of interest in a cancer stem cell marker.

The present invention provides methods of identifying a human subject for treatment with an agent described herein, comprising determining if the subject has a tumor that has an elevated level of B7-H4 as compared to expression of B7-H4 in normal tissue of the same type. In some embodiments, if the tumor has an elevated level of B7-H4, the subject is selected for treatment with an agent that specifically disrupts the binding of VSTM4 to B7-H4. In some embodiments, if selected for treatment, the subject is administered an agent described herein. In some embodiments, the subject has been previously treated with an anti-cancer therapeutic agent. In certain embodiments, the subject has had a tumor removed.

The present invention also provides methods of identifying a human subject for treatment with a binding agent, comprising determining if the subject has a tumor that has an aberrant expression of B7-H4 as compared to expression of B7-H4 in tissue of the same type. In some embodiments, if the tumor has an aberrant expression of B7-H4, the subject is selected for treatment with an agent that specifically disrupts the binding of VSTM4 to B7-H4. In some embodiments, if selected for treatment, the subject is administered an agent described herein. In some embodiments, the subject has been previously treated with an anti-cancer therapeutic agent. In certain embodiments, the subject has had a tumor removed.

The present invention also provides methods of selecting a human subject for treatment with an agent described herein, the method comprising determining if the subject has a tumor that has an elevated expression level of B7-H4, wherein if the tumor has an elevated expression level of B7-H4 the subject is selected for treatment. In some embodiments, a method of inhibiting tumor growth in a human subject comprises determining if the tumor has an elevated expression level of B7-H4, and administering to the subject a therapeutically effective amount of an agent described herein. In some embodiments, a method of treating cancer in a human subject comprises (a) selecting a subject for treatment based, at least in part, on the subject having a cancer that has an elevated level of B7-H4, and (b) administering to the subject a therapeutically effective amount of an agent described herein.

Methods for determining the level of nucleic acid expression in a cell, tumor, or cancer are known by those of skill in the art. These methods include, but are not limited to, PCR-based assays, microarray analyses, and nucleotide sequencing (e.g., NextGen sequencing). Methods for determining the level of protein expression in a cell, tumor, or cancer include, but are not limited to, Western blot analysis, protein arrays, ELISAs, immunohistochemistry (IHC), and FACS.

Methods for determining whether a tumor or cancer has an elevated level of expression of a nucleic acid or protein can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a blood sample. In some embodiments, the sample is a plasma sample. In some embodiments, the sample is processed to a cell lysate. In some embodiments, the sample is processed to DNA or RNA.

The present invention further provides pharmaceutical compositions comprising the binding agents described herein. In certain embodiments, the pharmaceutical compositions further comprise a pharmaceutically acceptable vehicle. In some embodiments, the pharmaceutical compositions find use in immunotherapy. In some embodiments, the pharmaceutical compositions find use in inhibiting tumor growth in a subject (e.g., a human patient). In some embodiments, the pharmaceutical compositions find use in treating cancer in a subject (e.g., a human patient).

In certain embodiments, formulations are prepared for storage and use by combining a purified binding agent of the present invention with a pharmaceutically acceptable vehicle (e.g., a carrier or excipient). Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22^{st} Edition, 2012, Pharmaceutical Press, London.).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The binding agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{st} Edition, 2012, Pharmaceutical Press, London.

In certain embodiments, pharmaceutical formulations include a binding agent of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations can be produced. Suitable examples of sustained-release preparations include semi-permeable matrices of solid hydrophobic polymers containing a binding agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In certain embodiments, in addition to administering a binding agent, the method or treatment further comprises administering at least one immune response stimulating agent. In some embodiments, the immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), an antibody that blocks immunosuppressive functions (e.g., an anti-CTLA4 antibody, anti-CD28 antibody, anti-PD1, anti-CD3 antibody), a toll-like receptor (e.g., TLR4, TLR7, TLR9), a member of the TNF receptor superfamily (e.g., OX40 (CD134), OX40L, glucocorticoid-induced TNFR-related (GITR) protein, or GITRL), or a member of the B7 family (e.g., CD80, CD86). In some embodiments, a method comprises administering an antibody that specifically binds VSTM4 and an antibody that specifically binds CTLA4. In some embodiments, a method comprises administering an antibody that specifically binds VSTM4 and an antibody that specifically binds PD1. In some embodiments, a method comprises administering an antibody that specifically binds VSTM4 and a polypeptide comprising OX40. In some embodiments, a method comprises administering an antibody that specifically binds VSTM4 and a polypeptide comprising OX40L. In some embodiments, a method comprises administering an antibody that specifically binds VSTM4 in combination with a polypeptide comprising GITR. In some embodiments, a method comprises administering an antibody that specifically binds VSTM4 in combination with a polypeptide comprising GITRL. In some embodiments, a method comprises administering an antibody that specifically binds VSTM4 in combination with IL-2. In some embodiments, a method comprises administering an antibody that specifically binds VSTM4 in combination with IL-15.

An immune response stimulating agent can be administered prior to, concurrently with, and/or subsequently to, administration of the binding agent. Pharmaceutical compositions comprising a binding agent and the immune response stimulating agent(s) are also provided. In some embodiments, the immune response stimulating agent comprises 1, 2, 3, or more immune response stimulating agents.

In certain embodiments, in addition to administering a binding agent, the method or treatment further comprises administering at least one additional therapeutic agent. An additional therapeutic agent can be administered prior to, concurrently with, and/or subsequently to, administration of the binding agent. Pharmaceutical compositions comprising a binding agent and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments, the combination of a binding agent and at least one additional therapeutic agent results in additive or synergistic results. In some embodiments, the combination therapy results in an increase in the therapeutic index of the binding agent. In some embodiments, the combination therapy results in an increase in the therapeutic index of the additional agent(s). In some embodiments, the combination therapy results in a decrease in the toxicity and/or side effects of the binding agent. In some embodiments, the combination therapy results in a decrease in the toxicity and/or side effects of the additional agent(s).

Useful classes of therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Therapeutic agents that may be administered in combination with the binding agents described herein include chemotherapeutic agents. Thus, in some embodiments, the method or treatment involves the administration of a binding agent of the present invention in combination with a chemotherapeutic agent or in combination with a cocktail of chemotherapeutic agents. Treatment with a binding agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4^{th} Edition, 2008, M. C. Perry, Editor, Lippincott, Williams & Wilkins, Philadelphia, Pa.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel.

In some embodiments, an additional therapeutic agent comprises an agent such as a small molecule. For example, treatment can involve the combined administration of a binding agent of the present invention with a small molecule that acts as an inhibitor against tumor-associated antigens including, but not limited to, EGFR, HER2 (ErbB2), and/or VEGF. In some embodiments, a binding agent of the present invention is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In certain embodiments, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the mTOR/AKR pathway.

In some embodiments, an additional therapeutic agent comprises a biological molecule, such as an antibody. For example, treatment can involve the combined administration of a binding agent of the present invention with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits β-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

Furthermore, treatment with a binding agent described herein can include combination treatment with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician.

In some embodiments, the binding agent can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β, TNF-α, VEGF, P1GF, IL-1, IL-2, IL-3, IL-4, IL-5, IL-6, IL-7, IL-12, IL-15, and IL-18.

In certain embodiments, the treatment involves the administration of a binding agent of the present invention in combination with radiation therapy. Treatment with a binding agent can occur prior to, concurrently with, or subsequent to administration of radiation therapy. Dosing schedules for such radiation therapy can be determined by the skilled medical practitioner.

In certain embodiments, the treatment involves the administration of a binding agent of the present invention in combination with anti-viral therapy. Treatment with a binding agent can occur prior to, concurrently with, or subsequent to administration of antiviral therapy. The anti-viral drug used in combination therapy will depend upon the virus the subject is infected with.

Combined administration can include co-administration, either in a single pharmaceutical formulation or using separate formulations, or consecutive administration in either order but generally within a time period such that all active agents can exert their biological activities simultaneously.

It will be appreciated that the combination of a binding agent and at least one additional therapeutic agent may be administered in any order or concurrently. In some embodiments, the binding agent will be administered to patients that have previously undergone treatment with a second therapeutic agent. In certain other embodiments, the binding agent and a second therapeutic agent will be administered substantially simultaneously or concurrently. For example, a subject may be given a binding agent (e.g., a soluble receptor) while undergoing a course of treatment with a second therapeutic agent (e.g., chemotherapy). In certain embodiments, a binding agent will be administered within 1 year of the treatment with a second therapeutic agent. In certain alternative embodiments, a binding agent will be administered within 10, 8, 6, 4, or 2 months of any treatment with a second therapeutic agent. In certain other embodiments, a binding agent will be administered within 4, 3, 2, or 1 weeks of any treatment with a second therapeutic agent. In some embodiments, a binding agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

For the treatment of a disease, the appropriate dosage of an agent of the present invention depends on the type of disease to be treated, the severity and course of the disease, the responsiveness of the disease, whether the binding agent is administered for therapeutic or preventative purposes, previous therapy, the patient's clinical history, and so on, all at the discretion of the treating physician. The agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In certain embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the binding agent is from about 0.1 mg to about 20 mg/kg of body weight. In some embodiments, the dosage of the binding agent is about 0.5 mg/kg of body weight. In some embodiments, the dosage of the binding agent is about 1 mg/kg of body weight. In some embodiments, the dosage of the binding agent is about 1.5 mg/kg of body weight. In some embodiments, the dosage of the binding agent is about 2 mg/kg of body weight. In some embodiments, the dosage of the binding agent is about 2.5 mg/kg of body weight. In some embodiments, the dosage of the binding agent is about 5 mg/kg of body weight. In some embodiments, the dosage of the binding agent is about 7.5 mg/kg of body weight. In some embodiments, the dosage of the binding agent is about 10 mg/kg of body weight. In some embodiments, the dosage of the binding agent is about 12.5 mg/kg of body weight. In some embodiments, the dosage of the binding agent is about 15 mg/kg of body weight. In certain embodiments, the dosage can be given once or more daily, weekly, monthly, or yearly. In certain embodiments, the binding agent is given once every week, once every two weeks, once every three weeks, or once every four weeks.

In some embodiments, an agent may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

Thus, the present invention provides methods of administering to a subject the binding agents described herein comprising using an intermittent dosing strategy for administering one or more agents, which may reduce side effects and/or toxicities associated with administration of a binding agent, chemotherapeutic agent, etc. In some embodiments, a method for treating cancer in a human subject comprises administering to the subject a therapeutically effective dose of a binding agent in combination with a therapeutically effective dose of a chemotherapeutic agent, wherein one or both of the agents are administered according to an intermittent dosing strategy. In some embodiments, the intermittent dosing strategy comprises administering an initial dose of a binding agent to the subject, and administering subsequent doses of the binding agent about once every 2 weeks. In some embodiments, the intermittent dosing strategy comprises administering an initial dose of a binding agent to the subject, and administering subsequent doses of the binding agent about once every 3 weeks. In some embodiments, the intermittent dosing strategy comprises administering an initial dose of a binding agent to the subject, and administering subsequent doses of the binding agent about once every 4 weeks. In some embodiments, the binding agent is administered using an intermittent dosing strategy and the chemotherapeutic agent is administered weekly.

### V. Screening

The present invention provides screening methods to identify agents that modulate the immune response. In some embodiments, the present invention provides methods for screening candidate agents, including but not limited to, proteins, antibodies, peptides, peptidomimetics, small molecules, compounds, or other drugs, which modulate the immune response.

In some embodiments, a method of screening for a candidate agent that modulates the immune response comprises determining if the agent has an effect on immune response cells. In some embodiments, a method of screening for a candidate agent that modulates the immune response comprises determining if the agent is capable of increasing the activity of immune cells. In some embodiments, a method of screening for a candidate agent that modulates the immune response comprises determining if the agent is capable of increasing the activity of cytolytic cells, such as CTLs and/or NK cells. In some embodiments, a method of screening for a candidate agent that modulates the immune response comprises determining if the agent is capable of inhibiting or blocking a B7-H4/VSTM4 signaling pathway.

### VI. Kits Comprising Binding Agents

The present invention provides kits that comprise the binding agents described herein and that can be used to perform the methods described herein. In certain embodiments, a kit comprises at least one purified binding agent in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed binding agents of the present invention can be readily incorporated into one of the established kit formats which are well known in the art.

Further provided are kits that comprise a binding agent as well as at least one additional therapeutic agent. In certain embodiments, the second (or more) therapeutic agent is a chemotherapeutic agent.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Binding Interactions Between B7-H4 and VSTM4

The binding interactions B7-H4 and potential receptors were examined by flow cytometry. As Previous attempts by researchers to identify a B7-H4 receptor have failed. The inventors hypothesized that the affinity of the receptor-ligand interaction may be low and that commonly used methods had missed the interaction between B7-H4 and its receptor due to weak binding. To overcome this potential issue, a fusion protein strategy that presented five B7-H4 proteins within a pentameric molecule that utilized the multimerization motif of the C-terminal cartilage oligomeric matrix protein (COMP). The rationale was that the ability to enable greater avidity in B7-H4 molecule may facilitate the observation of otherwise weak binding interactions between B7-H4 and its receptor.

The B7-H4-COMP-FLAG fusion protein was produced using an expression vector containing a sequence encoding the extracellular N-terminal domain of human B7-H4 fused to a sequence encoding the multimerization domain of murine C-terminal cartilage oligomeric matrix protein and a FLAG epitope tag (COMP-FLAG) using methods previously described (Voulgaraki et al., 2005, Immunology, 115:337-346). The construct produced secreted pentamers of the extracellular domain of human B7-H4. Briefly, the B7-H4-COMP-FLAG encoding expression vector was transfected into HEK-293T cells using Lipofectamine 2000 (Life Technologies Inc.) and conditioned media was collected 72 hours later. B7-H4-COMP-FLAG proteins were purified using the ANTI-FLAG M2 Affinity gel (Sigma-Aldrich, St. Louis Mo.) following the manufacturer's instructions.

Candidate receptor proteins were expressed in a membrane-anchored form containing the extracellular domain of the receptor, or a fragment thereof, fused to a human CD4 transmembrane region and an intracellular green fluorescent protein (GFP) tag.

HEK-293T cells were transiently transfected with an expression vector encoding each of the candidate receptors. Transfected cells were subsequently contacted with various concentrations (0 μg/ml, 0.5 μg/ml, 2 μg/ml, or 8 μg/ml) of the B7-H4-COMP-FLAG protein in staining media (PBS/2% FBS). The cells were incubated for one hour at 4° C. Cells were subsequently washed with staining media and incubated for 30 minutes at 4° C. with an anti-FLAG antibody conjugated to Alexa647 (Cell Signaling Technology) to identify the cells bound by B7-H4-COMP-FLAG. The cells were washed again and resuspended in staining media containing 1 μg/ml DAPI. FACS analysis was performed using a CANTO II instrument (BD Biosciences, San Jose, Calif.) and the data analyzed using FlowJo software. Binding is indicated by the detection of cells within the inset box.

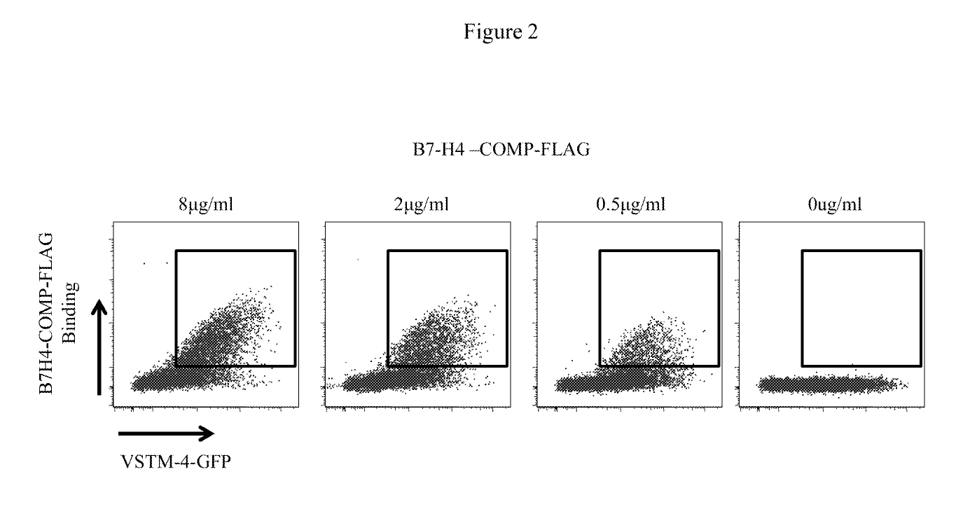

As is shown in FIG. 2, human B7-H4 was found to bind human VSTM4 in a dose-dependent manner.

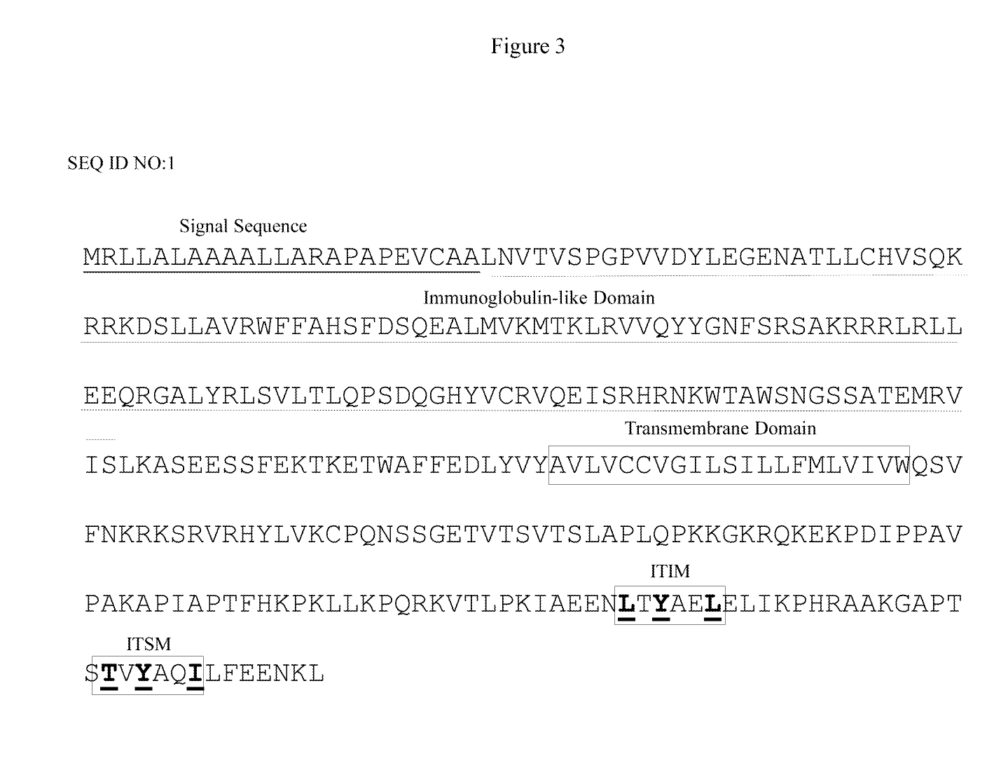

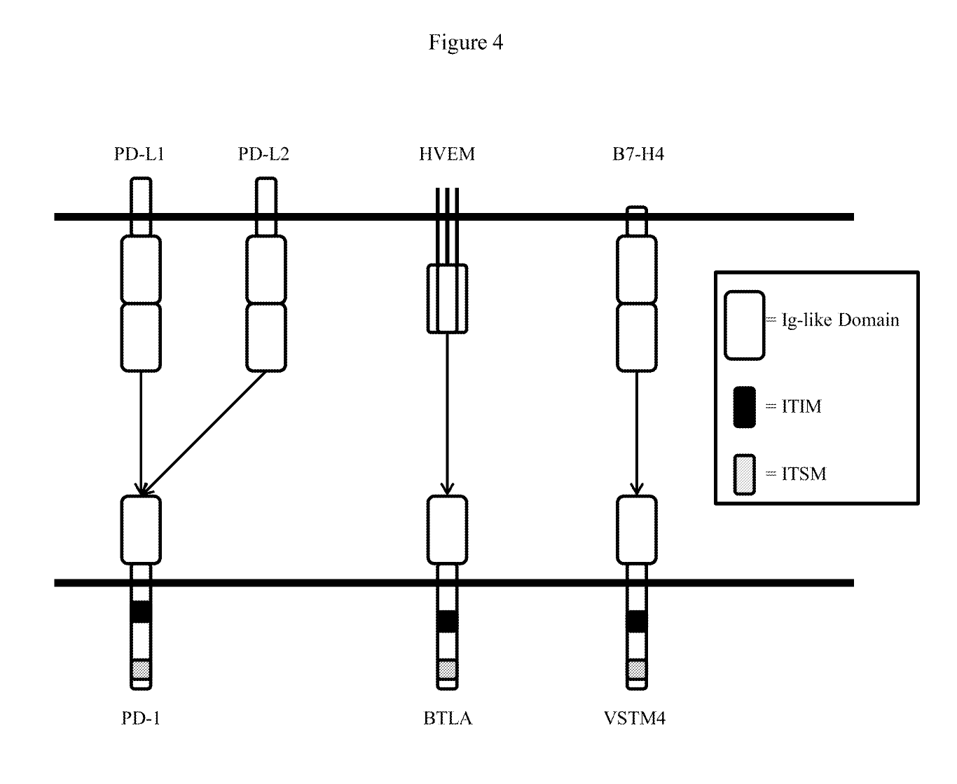

VSTM4 is part of the large Ig superfamily and is predicted to have a single extracellular Ig-like domain. FIG. 3 shows the sequence of human VSTM4 with a number of important motifs marked. VSTM4 has structural features that suggest it may function as an inhibitory receptor, including an immunoreceptor tyrosine-based inhibitory motif (ITIM) and an immunoreceptor tyrosine-based switch motif (ITSM) within the predicted intracellular domain. The consensus sequence for ITIM is (S/I/V/L)LXYXX(I/V/L) (SEQ ID NO:38) and the consensus sequence for ITSM is TXYXX(V/I) (SEQ ID NO:39), wherein X is any amino acid residue. Overall this structure bears similarity to PD-1 and BTLA which each possess an ITIM and an ITSM within their intracellular domain (see FIG. 4). Furthermore, PD-1, BTLA, and VSTM4 each possess a similar structure with the ITSM residing C-terminal to the ITIM. These motifs are able to recruit phosphatases and inhibit signaling. These structural characteristics suggest that VSTM4 is an inhibitory receptor and a potentially important immune checkpoint.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

Following are the sequences disclosed in the application:

Human VSTM4 with predicted signal sequence
underlined
(SEQ ID NO: 1)
MRLLALAAAALLARAPAPEVCAALNVTVSPGPVVDYLEGENATLLCHVSQ
KRRKDSLLAVRWFFAHSFDSQEALMVKMTKLRVVQYYGNFSRSAKRRRLR
LLEEQRGALYRLSVLTLQPSDQGHYVCRVQEISRHRNKWTAWSNGSSATE
MRVISLKASEESSFEKTKETWAFFEDLYVYAVLVCCVGILSILLFMLVIV
WQSVFNKRKSRVRHYLVKCPQNSSGETVTSVTSLAPLQPKKGKRQKEKPD
IPPAVPAKAPIAPTFHKPKLLKPQRKVTLPKIAEENLTYAELELIKPHRA
AKGAPTSTVYAQILFEENKL
Human VSTM4 ECD(aa 1-180) with predicted signal
sequence underlined
(SEQ ID NO: 2)
MRLLALAAAALLARAPAPEVCAALNVTVSPGPVVDYLEGENATLLCHVSQ
KRRKDSLLAVRWFFAHSFDSQEALMVKMTKLRVVQYYGNFSRSAKRRRLR
LLEEQRGALYRLSVLTLQPSDQGHYVCRVQEISRHRNKWTAWSNGSSATE
MRVISLKASEESSFEKTKETWAFFEDLYVY
Human VSTM4 ECD(aa 24-180) without predicted
signal sequence
(SEQ ID NO: 3)
LNVTVSPGPVVDYLEGENATLLCHVSQKRRKDSLLAVRWFFAHSFDSQEA
LMVKMTKLRVVQYYGNFSRSAKRRRLRLLEEQRGALYRLSVLTLQPSDQG
HYVCRVQEISRHRNKWTAWSNGSSATEMRVISLKASEESSFEKTKETWAF
FEDLYVY
Human VSTM4 Ig-like domain (aa 24-155)
(SEQ ID NO: 4)
LNVTVSPGPVVDYLEGENATLLCHVSQKRRKDSLLAVRWFFAHSFDSQEA
LMVKMTKLRVVQYYGNFSRSAKRRRLRLLEEQRGALYRLSVLTLQPSDQG
HYVCRVQEISRHRNKWTAWSNGSSATEMRVIS
Human VSTM4 IgV domain (aa 24-130)
(SEQ ID NO: 5)
LNVTVSPGPVVDYLEGENATLLCHVSQKRRKDSLLAVRWFFAHSFDSQEA
LMVKMTKLRVVQYYGNFSRSAKRRRLRLLEEQRGALYRLSVLTLQPSDQG
HYVCRVQ
Mouse VSTM4 with predicted signal sequence
underlined
(SEQ ID NO: 6)
MRLRLLALAAAVLLGPAPEVCGALNVTVSPGPVVDYLEGENATLLCHVSQ
KRRKDSLLAVRWFFAPDGSQEALMVKMTKLRIIQYYGNFSRTANQQRLRL
LEERRGVLYRLSVLTLRPTDQGQYVCKVQEISKHRNKWTAWSNGSSATEM
RVISLKAGEDSSFEKKKVTWAFFEDLYVYAVLVCCVGILSVLLFTLVIAW
QSVFHKRKSRVRHYLVKCPQNSSGETVTSVTSLAPLQPQKGKRQKKKVDV
PPAVPAKAPIATTFHKPKLLKPQRKVALPKITEENLTYAELELIKPHRAA
KGVPTSTVYAQILFEENQL
Mouse VSTM4 ECD(aa 1-179) with predicted signal
sequence underlined
(SEQ ID NO: 7)
MRLRLLALAAAVLLGPAPEVCGALNVTVSPGPVVDYLEGENATLLCHVSQ
KRRKDSLLAVRWFFAPDGSQEALMVKMTKLRIIQYYGNFSRTANQQRLRL
LEERRGVLYRLSVLTLRPTDQGQYVCKVQEISKHRNKWTAWSNGSSATEM
RVISLKAGEDSSFEKKKVTWAFFEDLYVY
Mouse VSTM4 ECD(aa 24-179) without predicted
signal sequence
(SEQ ID NO: 8)
LNVTVSPGPVVDYLEGENATLLCHVSQKRRKDSLLAVRWFFAPDGSQEAL
MVKMTKLRIIQYYGNFSRTANQQRLRLLEERRGVLYRLSVLTLRPTDQGQ
YVCKVQEISKHRNKWTAWSNGSSATEMRVISLKAGEDSSFEKKKVTWAFF
EDLYVY
Mouse VSTM4 Ig-like domain (aa 24-154)
(SEQ ID NO: 9)
LNVTVSPGPVVDYLEGENATLLCHVSQKRRKDSLLAVRWFFAPDGSQEAL
MVKMTKLRIIQYYGNFSRTANQQRLRLLEERRGVLYRLSVLTLRPTDQGQ
YVCKVQEISKHRNKWTAWSNGSSATEMRVIS
Mouse VSTM4 Ig-like V domain (aa 24-129)
(SEQ ID NO: 10)
LNVTVSPGPVVDYLEGENATLLCHVSQKRRKDSLLAVRWFFAPDGSQEAL
MVKMTKLRIIQYYGNFSRTANQQRLRLLEERRGVLYRLSVLTLRPTDQGQ
YVCKVQ
Human B7-H4 with predicted signal sequence
underlined
(SEQ ID NO: 11)
MASLGQILFWSIISIIIILAGAIALIIGFGISGRHSITVTTVASAGNIGE
DGILSCTFEPDIKLSDIVIQWLKEGVLGLVHEFKEGKDELSEQDEMFRGR
TAVFADQVIVGNASLRLKNVQLTDAGTYKCYIITSKGKGNANLEYKTGAF
SMPEVNVDYNASSETLRCEAPRWFPQPTVVWASQVDQGANFSEVSNTSFE
LNSENVTMKVVSVLYNVTINNTYSCMIENDIAKATGDIKVTESEIKRRSH
LQLLNSKASLCVSSFFAISWALLPLSPYLMLK
Human B7-H4 ECD(aa 1-259) with predicted signal
sequence underlined
(SEQ ID NO: 12)
MASLGQILFWSIISIIIILAGAIALIIGFGISGRHSITVTTVASAGNIGE
DGILSCTFEPDIKLSDIVIQWLKEGVLGLVHEFKEGKDELSEQDEMFRGR
TAVFADQVIVGNASLRLKNVQLTDAGTYKCYIITSKGKGNANLEYKTGAF
SMPEVNVDYNASSETLRCEAPRWFPQPTVVWASQVDQGANFSEVSNTSFE
LNSENVTMKVVSVLYNVTINNTYSCMIENDIAKATGDIKVTESEIKRRSH
LQLLNSKAS
Human B7-H4 ECD(aa 25-259) without predicted
signal sequence
(SEQ ID NO: 13)
LIIGFGISGRHSITVTTVASAGNIGEDGILSCTFEPDIKLSDIVIQWLKE
GVLGLVHEFKEGKDELSEQDEMFRGRTAVFADQVIVGNASLRLKNVQLTD
AGTYKCYIITSKGKGNANLEYKTGAFSMPEVNVDYNASSETLRCEAPRWF
PQPTVVWASQVDQGANFSEVSNTSFELNSENVTMKVVSVLYNVTINNTYS
CMIENDIAKATGDIKVTESEIKRRSHLQLLNSKAS
Human B7-H4 Ig-like V domain 1 (aa 35-146)
(SEQ ID NO: 14)
HSITVTTVASAGNIGEDGILSCTFEPDIKLSDIVIQWLKEGVLGLVHEFK
EGKDELSEQDEMFRGRTAVFADQVIVGNASLRLKNVQLTDAGTYKCYIIT
SKGKGNANLEYK
Human B7-H4 Ig-like V domain 2 (aa 153-241)
(SEQ ID NO: 15)
PEVNVDYNASSETLRCEAPRWFPQPTVVWASQVDQGANFSEVSNTSFELN
SENVTMKVVSVLYNVTINNTYSCMIENDIAKATGDIKVT
Mouse B7-H4 with predicted signal sequence
underlined
(SEQ ID NO: 16)
MASLGQIIFWSIINIIIILAGAIALIIGFGISGKHFITVTTFTSAGNIGE
DGTLSCTFEPDIKLNGIVIQWLKEGIKGLVHEFKEGKDDLSQQHEMFRGR
TAVFADQVVVGNASLRLKNVQLTDAGTYTCYIRTSKGKGNANLEYKTGAF
SMPEINVDYNASSESLRCEAPRWFPQPTVAWASQVDQGANFSEVSNTSFE
LNSENVTMKVVSVLYNVTINNTYSCMIENDIAKATGDIKVTDSEVKRRSQ
LQLLNSGPSPCVFSSAFVAGWALLSLSCCLMLR
Mouse B7-H4 ECD(aa 1-257) with predicted signal
sequence underlined
(SEQ ID NO: 17)
MASLGQIIFWSIINIIIILAGAIALIIGFGISGKHFITVTTFTSAGNIGE
DGTLSCTFEPDIKLNGIVIQWLKEGIKGLVHEFKEGKDDLSQQHEMFRGR
TAVFADQVVVGNASLRLKNVQLTDAGTYTCYIRTSKGKGNANLEYKTGAF
SMPEINVDYNASSESLRCEAPRWFPQPTVAWASQVDQGANFSEVSNTSFE
LNSENVTMKVVSVLYNVTINNTYSCMIENDIAKATGDIKVTDSEVKRRSQ
LQLLNSG
Mouse B7-H4 ECD(aa 25-257) without predicted
signal sequence
(SEQ ID NO: 18)
LIIGFGISGKHFITVTTFTSAGNIGEDGTLSCTFEPDIKLNGIVIQWLKE
GIKGLVHEFKEGKDDLSQQHEMFRGRTAVFADQVVVGNASLRLKNVQLTD
AGTYTCYIRTSKGKGNANLEYKTGAFSMPEINVDYNASSESLRCEAPRWF
PQPTVAWASQVDQGANFSEVSNTSFELNSENVTMKVVSVLYNVTINNTYS
CMIENDIAKATGDIKVTDSEVKRRSQLQLLNSG
Mouse B7-H4 Ig-like V-type domain 1 (aa 35-144)
(SEQ ID NO: 19)
KHFITVTTFTSAGNIGEDGTLSCTFEPDIKLNGIVIQWLKEGIKGLVHEF
KEGKDDLSQQHEMFRGRTAVFADQVVVGNASLRLKNVQLTDAGTYTCYIR
TSKGKGNANLE
Mouse B7-H4 Ig-like V-type domain 2 (aa 153-241)
(SEQ ID NO: 20)
WFPQPTVAWASQVDQGANFSEVSNTSFELNSENVTMKVVSVLYNVTINNT
YSCMIENDIAKATGDIKVT
Human IgG₁ Fc region
(SEQ ID NO: 21)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK
Human IgG₁ Fc region
(SEQ ID NO: 22)
KSSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG₁ Fc region
(SEQ ID NO: 23)
EPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK
Human IgG2 Fc region
(SEQ ID NO: 24)
CVECPPCPAPPVAGPSVFLFPPKPKDTLMISRTPEVTCVVVDVSHEDPEV
QFNWYVDGVEVHNAKTKPREEQFNSTFRVVSVLTVVHQDWLNGKEYKCKV
SNKGLPAPIEKTISKTKGQPREPQVYTLPPSREEMTKNQVSLTCLVKGFY
PSDIAVEWESNGQPENNYKTTPPMLDSDGSFFLYSKLTVDKSRWQQGNVF
SCSVMHEALHNHYTQKSLSLSPGK
Mouse IgG1 Fc region
(SEQ ID NO: 25)
GCKPCICTVPEVSSVFIFPPKPKDVLTITLTPKVTCVVVDISKDDPEVQF
SWFVDDVEVHTAQTQPREEQFNSTFRSVSELPIMHQDWLNGKEFKCRVNS
AAFPAPIEKTISKTKGRPKAPQVYTIPPPKEQMAKDKVSLTCMITDFFPE
DITVEWQWNGQPAENYKNTQPIMDTDGSYFVYSKLNVQKSNWEAGNTFTC
SVLHEGLHNHHTEKSLSHSPGK
Mouse IgG2a Fc region
(SEQ ID NO: 26)
IKPCPPCKCPAPNLLGGPSVFIFPPKIKDVLMISLSPIVTCVVVDVSEDD
PDVQISWFVNNVEVHTAQTQTHREDYNSTLRVVSALPIQHQDWMSGKEFK
CKVNNKDLPAPIERTISKPKGSVRAPQVYVLPPPEEEMTKKQVTLTCMVT
DFMPEDIYVEWTNNGKTELNYKNTEPVLDSDGSYFMYSKLRVEKKNWVER
NSYSCSVVHEGLHNHHTTKSFSRTPGK
Human IgG1 Heavy chain constant region
(SEQ ID NO: 27)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG2 Heavy chain constant region
(SEQ ID NO: 28)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
Human IgG3 Heavy chain constant region
(SEQ ID NO: 29)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK
Human IgG4 Heavy chain constant region
(SEQ ID NO: 30)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
FLAGTag
(SEQ ID NO: 31)
DYKDDDDK
Linker
(SEQ ID NO: 32)
ESGGGGVT
Linker
(SEQ ID NO: 33)
LESGGGGVT
Linker
(SEQ ID NO: 34)
GRAQVT
Linker
(SEQ ID NO: 35)
WRAQVT
Linker
(SEQ ID NO: 36)
ARGRAQVT
ITAM
(SEQ ID NO: 37)
YXX(L/I)(X)₍₆₋₈₎YXX(L/I)
wherein X is any amino acid residue
ITIM
(SEQ ID NO: 38)
(S/I/V/L)LXYXX(I/V/L)
ITSM
(SEQ ID NO: 39)
TXYXX(V/I)
wherein X is any amino acid residue

What is claimed is:

1. An antibody that specifically binds the extracellular domain of V-set and transmembrane domain-containing protein 4 (VSTM4).

2. The antibody of claim 1, wherein the VSTM4 is human VSTM4, mouse VSTM4, or human and mouse VSTM4.

3. The antibody of claim 1 or claim 2, which binds the Ig-like domain of VSTM4.

4. The antibody of claim 1 or claim 2, which binds the Ig V-type domain of VSTM4.

5. The antibody of claim 1 or claim 2, which binds within amino acids 24-155 of human VSTM4 or mouse VSTM4.

6. The antibody of claim 1 or claim 2, which binds within amino acids 24-130 of human VSTM4 or mouse VSTM4.

7. The antibody of claim 1 or claim 2, which binds within SEQ ID NO:3, SEQ ID NO:4, SEQ ID NO:5, SEQ ID NO:8, SEQ ID NO:9, or SEQ ID NO:10.

8. The antibody of any one of claims 1 to 7, which is a monoclonal antibody, a chimeric antibody, a humanized antibody, a human antibody, a bispecific antibody, or an antibody fragment.

9. A soluble receptor comprising the extracellular domain of VSTM4 or a fragment thereof.

10. The soluble receptor of claim 9, wherein the VSTM4 is human VSTM4 or mouse VSTM4.

11. The soluble receptor of claim 9 or claim 10, comprising the Ig-like domain of VSTM4.

12. The soluble receptor of claim 9 or claim 10, comprising the Ig V-type domain of VSTM4.

13. The soluble receptor of claim 9 or claim 10, comprising amino acids 24-155 of human VSTM4 or 24-154 of mouse VSTM4.

14. The soluble receptor of claim 9 or claim 10, wherein the soluble receptor comprises amino acids 24-130 of human VSTM4 or 24-129 of mouse VSTM4.

15. The soluble receptor of claim 9 or claim 10, wherein the soluble receptor comprises SEQ ID NO:3, SEQ ID NO:4, SEQ ID NO:5, SEQ ID NO:8, SEQ ID NO:9, SEQ ID NO:10, or a fragment thereof.

16. The soluble receptor of any one of claims 9 to 15, which further comprises a non-VSTM4 polypeptide.

17. The soluble receptor of claim 16, wherein the non-VSTM4 polypeptide comprises a Fc region.

18. The soluble receptor of claim 17, wherein the Fc region is selected from the group consisting of SEQ ID NO:21, SEQ ID NO:22, SEQ ID NO:23, SEQ ID NO:24, SEQ ID NO: 25 and SEQ ID NO:26.

19. An isolated polynucleotide comprising a polynucleotide that encodes the antibody or the soluble receptor of any one of claims 1 to 18.

20. A vector comprising the polynucleotide of claim 19.

21. A cell comprising the polynucleotide of claim 19 or the vector of claim 20.

22. A cell producing the antibody or the soluble receptor of any one of claims 1 to 18.

23. A composition comprising the antibody or the soluble receptor of any one of claims 1 to 18.

24. A pharmaceutical composition comprising the antibody or the soluble receptor of any one of claims 1 to 18 and a pharmaceutically acceptable carrier.

25. The antibody or soluble receptor of any one of claims 1 to 18, which disrupts signaling of a B7-H4/VSTM4 pathway.

26. The antibody or soluble receptor of any one of claims 1 to 18, which:
(a) disrupts binding of VSTM4 to B7-H4; and/or
(b) disrupts B7-H4 activation of VSTM4 signaling.

27. A method of inducing, activating, promoting, increasing, enhancing, or prolonging an immune response in a subject, comprising administering a therapeutically effective amount of an antibody or a soluble receptor of any one of claims 1 to 18.

28. The method of claim 27, wherein the immune response is against a tumor or cancer.

29. The method of claim 27, wherein the immune response is against a viral infection.

30. A method of inhibiting growth of a tumor, comprising contacting a tumor or tumor cell with an effective amount of an antibody or a soluble receptor of any one of claims 1 to 18.

31. A method of inhibiting growth of a tumor in a subject, comprising administering to the subject a therapeutically effective amount of an antibody or a soluble receptor of any one of claims 1 to 18.

32. A method of treating cancer in a subject, comprising administering to the subject a therapeutically effective amount of an antibody or a soluble receptor of any one of claims 1 to 18.

33. A method of stimulating a protective immune response, comprising administering an antibody or soluble receptor of any one of claims 1 to 18 in combination with an antigen of interest.

34. The method of claim 33, wherein the antigen of interest is a tumor antigen.

35. An agent that specifically binds the extracellular domain of VSTM4, wherein the agent disrupts signaling of a B7-H4/VSTM4 pathway.

36. An agent that specifically binds the extracellular domain of B7-H4, wherein the agent disrupts signaling from a B7-H4/VSTM4 pathway.

37. An agent that specifically binds the extracellular domain of VSTM4, wherein the agent:
(a) disrupts binding of VSTM4 to B7-H4; and/or
(b) disrupts B7-H4 activation of VSTM4 signaling.

38. An agent that specifically binds the extracellular domain of B7-H4, wherein the agent:
(a) disrupts binding of B7-H4 to VSTM4; and/or
(b) disrupts B7-H4 activation of VSTM4 signaling.

39. The agent of any one of claims 35 to 38, which disrupts binding of VSTM4 to B7-H4.

40. The agent of any one of claims 35 to 38, which disrupts binding of B7-H4 to VSTM4.

41. The agent of any one of claims 35 to 40, which disrupts B7-H4 activation of VSTM4 signaling.

42. The agent of any one of claims 35 to 40, which induces, activates, promotes, increases, enhances, or prolongs an immune response.

43. The agent of any one of claims 35 to 40, which inhibits activity of VSTM4.

44. The agent of any one of claims 35 to 40, which inhibits the inhibitory activity of VSTM4.

45. The agent of any one of claims 35 to 44, which is an antibody.

46. The agent of claim 45, wherein the antibody is a monoclonal antibody, a chimeric antibody, a humanized antibody, a human antibody, a bispecific antibody, or an antibody fragment.

47. The agent of any one of claims 35 to 44, which is a soluble receptor.

48. The agent of claim 47, wherein the soluble receptor comprises the extracellular domain of VSTM4 or a fragment thereof.

49. The agent of claim 47 or claim 48, wherein the soluble receptor comprises a Fc region.

50. The agent of any one of claims 35 to 49, which is an antagonist of VSTM4 signaling.

51. The agent of any one of claims 35 to 50, which increases cell-mediated immunity.

52. The agent of any one of claims 35 to 50, which increases T-cell activity.

53. The agent of any one of claims 35 to 50, which increases cytolytic T-cell (CTL) activity.

54. The agent of any one of claims 35 to 50, which increases natural killer (NK) activity.

55. A composition comprising the agent of any one of claims 35 to 50.

56. A pharmaceutical composition comprising the agent of any one of claims 35 to 50 and a pharmaceutically acceptable carrier.

57. An isolated polynucleotide comprising a polynucleotide that encodes the agent of any one of claims 35 to 50.

58. A vector comprising the polynucleotide of claim 57.

59. An isolated cell comprising the polynucleotide of claim 57 or the vector of claim 58.

60. An isolated cell producing the agent of any one of claims 35 to 54.

61. A method of inducing, activating, promoting, increasing, or enhancing an immune response in a subject, comprising administering to the subject a therapeutically effective amount of an agent of any one of claims 35 to 54.

62. A method of inducing, activating, promoting, increasing, or enhancing an immune response in a subject, comprising administering to the subject a therapeutically effective amount of an agent that disrupts the signaling of a B7-H4/VSTM4 pathway.

63. The method of claim 62, wherein the disruption of the signaling of a B7-H4/VSTM4 pathway inhibits the B7-H4-mediated suppression of immune responses.

64. The method of any one of claims 51 to 63, wherein the immune response is against a tumor or cancer.

65. The method of any one of claims 51 to 63, wherein the immune response is against a viral infection.

66. A method of inhibiting growth of a tumor, comprising contacting a tumor or tumor cell with an effective amount of an agent of any one of claims 35 to 54.

67. A method of inhibiting growth of a tumor in a subject, comprising administering to the subject a therapeutically effective amount of an agent of any one of claims 35 to 54.

68. A method of treating cancer in a subject, comprising administering a therapeutically effective amount of an agent of any one of claims 35 to 54.

69. A method of inhibiting growth of a tumor, comprising contacting a tumor or tumor cell with an effective amount of an agent that disrupts the interaction between B7-H4 and VSTM4.

70. A method of inhibiting growth of a tumor in a subject, comprising administering to the subject a therapeutically effective amount of an agent that disrupts the interaction between B7-H4 and VSTM4.

71. A method of treating cancer in a subject, comprising administering to the subject a therapeutically effective amount of an agent that disrupts the interaction between B7-H4 and VSTM4.

72. The method of any one of claims 69 to 71, wherein the agent specifically binds B7-H4.

73. The method of any one of claims 69 to 71, wherein the agent specifically binds VSTM4.

74. The method of any one of claims 61 to 73, further comprising administering a therapeutically effective amount of an antibody that specifically binds PD-1.

75. The method of any one of claims 61 to 73, further comprising administering a therapeutically effective amount of an antibody that specifically binds CTLA-4.

76. A method for activating anti-tumor immune responses in a subject, comprising administering a therapeutically effective amount of an agent of any one of claims 35 to 54.

77. A method to stimulate a protective immune response, comprising administering the agent of any one of claims 35-54 in combination with an antigen of interest.

78. The method of claim 77, wherein the antigen of interest is a tumor antigen.

79. The method of claim 77, wherein the antigen of interest is a cancer stem cell marker.
